SEQUENCE LISTING (46 sequences)

SEQ ID NO: 1 - DNA, 30 bp, synthetic construct
  source 1..30: mol_type = other DNA
tttactggagagacttgagactcgaacttt

SEQ ID NO: 2 - DNA, 30 bp, synthetic construct
  source 1..30: mol_type = other DNA
tggatgctagcaatggtaacgatcgtaggt

SEQ ID NO: 3 - DNA, 30 bp, synthetic construct
  source 1..30: mol_type = other DNA
ttttagtggtcctcagctactcgatgtttt

SEQ ID NO: 4 - DNA, 30 bp, synthetic construct
  source 1..30: mol_type = other DNA
tttttgccagcagtgagtcagaccgttttt

SEQ ID NO: 5 - DNA, 14 bp, synthetic construct
  source 1..14: mol_type = other DNA
agtctcaagtctct

SEQ ID NO: 6 - DNA, 16 bp, synthetic construct
  source 1..16: mol_type = other DNA
atcgttaccattgcta

SEQ ID NO: 7 - DNA, 14 bp, synthetic construct
  source 1..14: mol_type = other DNA
gagtagctgaggac

SEQ ID NO: 8 - DNA, 14 bp, synthetic construct
  source 1..14: mol_type = other DNA
tctgactcactgct

SEQ ID NO: 9 - DNA, 59 bp, synthetic construct
  source 1..59: mol_type = other DNA
gctgaggtggtgtcacacgtctgcggttttttccgcagacgtgtgacaccattagagac

SEQ ID NO: 10 - DNA, 28 bp, synthetic construct
  source 1..28: mol_type = other DNA
ctagcagtctctaatacctcagcgctag

SEQ ID NO: 11 - DNA, 48 bp, synthetic construct
  source 1..48: mol_type = other DNA
tttttttttttttttttttttttttttttttttgacaccattagagac

SEQ ID NO: 12 - DNA, 47 bp, synthetic construct
  source 1..47: mol_type = other DNA
gctgaggtggtgtcttttttttttttttttttttttttttttttttt

SEQ ID NO: 13 - DNA, 54 bp, synthetic construct
  source 1..54: mol_type = other DNA
gctgaggtggtgtcttttttttttttttttttttttttttgacaccttagagac

SEQ ID NO: 14 - DNA, 55 bp, synthetic construct
  source 1..55: mol_type = other DNA
gctgaggtggtgtcttttttttttttttttttttttttttgacaccattagagac

SEQ ID NO: 15 - DNA, 56 bp, synthetic construct
  source 1..56: mol_type = other DNA
gctgaggtggtgtcttttttttttttttttttttttttttgacaccaattagagac

SEQ ID NO: 16 - DNA, 55 bp, synthetic construct
  source 1..55: mol_type = other DNA
gctgaggtaggtgtcttttttttttttttttttttttttttgacaccttagagac

SEQ ID NO: 17 - DNA, 56 bp, synthetic construct
  source 1..56: mol_type = other DNA
gctgaggtaggtgtcttttttttttttttttttttttttttgacaccattagagac

SEQ ID NO: 18 - DNA, 57 bp, synthetic construct
  source 1..57: mol_type = other DNA
gctgaggtaggtgtcttttttttttttttttttttttttttgacaccaattagagac

SEQ ID NO: 19 - DNA, 56 bp, synthetic construct
  source 1..56: mol_type = other DNA
gctgaggtaaggtgtcttttttttttttttttttttttttttgacaccttagagac

SEQ ID NO: 20 - DNA, 57 bp, synthetic construct
  source 1..57: mol_type = other DNA
gctgaggtaaggtgtcttttttttttttttttttttttttttgacaccattagagac

SEQ ID NO: 21 - DNA, 58 bp, synthetic construct
  source 1..58: mol_type = other DNA
gctgaggtaaggtgtcttttttttttttttttttttttttttgacaccaattagagac

SEQ ID NO: 22 - DNA, 27 bp, synthetic construct
  source 1..27: mol_type = other DNA
ctagcagtctctaaacctcagcgctag

SEQ ID NO: 23 - DNA, 28 bp, synthetic construct
  source 1..28: mol_type = other DNA
ctagcagtctctaatacctcagcgctag

SEQ ID NO: 24 - DNA, 28 bp, synthetic construct
  source 1..28: mol_type = other DNA
ctagcagtctctaaaacctcagcgctag

SEQ ID NO: 25 - DNA, 29 bp, synthetic construct
  source 1..29: mol_type = other DNA
ctagcagtctctaattacctcagcgctag

SEQ ID NO: 26 - DNA, 29 bp, synthetic construct
  source 1..29: mol_type = other DNA
ctagcagtctctaaaaacctcagcgctag

SEQ ID NO: 27 - DNA, 38 bp, synthetic construct
  source 1..38: mol_type = other DNA
gctgaggtggtgtcttttttttttgacaccttagagac

SEQ ID NO: 28 - DNA, 36 bp, synthetic construct
  source 1..36: mol_type = other DNA
gctgaggtgtgtcttttttttttgacacttagagac

SEQ ID NO: 29 - DNA, 34 bp, synthetic construct
  source 1..34: mol_type = other DNA
gctgaggttgtcttttttttttgacattagagac

SEQ ID NO: 30 - DNA, 32 bp, synthetic construct
  source 1..32: mol_type = other DNA
gctgaggtgtcttttttttttgacttagagac

SEQ ID NO: 31 - DNA, 30 bp, synthetic construct
  source 1..30: mol_type = other DNA
gctgaggttcttttttttttgattagagac

SEQ ID NO: 32 - DNA, 28 bp, synthetic construct
  source 1..28: mol_type = other DNA
gctgaggtcttttttttttgttagagac

SEQ ID NO: 33 - DNA, 26 bp, synthetic construct
  source 1..26: mol_type = other DNA
gctgaggtttttttttttttagagac

SEQ ID NO: 34 - DNA, 29 bp, synthetic construct
  source 1..29: mol_type = other DNA
ctagcagtctctaattacctcagcgctag

SEQ ID NO: 35 - DNA, 26 bp, synthetic construct
  source 1..26: mol_type = other DNA
gctgaggtttttttttttttagagac

SEQ ID NO: 36 - DNA, 46 bp, synthetic construct
  source 1..46: mol_type = other DNA
ttagagacttttttttttttttttttttttttttttttgctgaggt

SEQ ID NO: 37 - DNA, 39 bp, synthetic construct
  source 1..39: mol_type = other DNA
atcggatcgcgttttttttttttttttttttttagagac

SEQ ID NO: 38 - DNA, 39 bp, synthetic construct
  source 1..39: mol_type = other DNA
cgcgatccgatttttttttttttttttttttgctgaggt

SEQ ID NO: 39 - DNA, 39 bp, synthetic construct
  source 1..39: mol_type = other DNA
ttagagacttttttttttttttttttttatcggatcgcg

SEQ ID NO: 40 - DNA, 39 bp, synthetic construct
  source 1..39: mol_type = other DNA
gctgaggtttttttttttttttttttttcgcgatccgat

SEQ ID NO: 41 - DNA, 19 bp, synthetic construct
  source 1..19: mol_type = other DNA
agtctctaattacctcagc

SEQ ID NO: 42 - DNA, 50 bp, synthetic construct
  source 1..50: mol_type = other DNA
tttttttttttttttttttttttttttttttttttgacaccattagagac

SEQ ID NO: 43 - DNA, 50 bp, synthetic construct
  source 1..50: mol_type = other DNA
gctgaggtaggtgtcttttttttttttttttttttttttttttttttttt

SEQ ID NO: 44 - DNA, 29 bp, synthetic construct
  source 1..29: mol_type = other DNA
ctagcagtctctaaaaacctcagcgctag

SEQ ID NO: 45 - DNA, 35 bp, synthetic construct
  source 1..35: mol_type = other DNA
ttttttttttttttttttttgacaccattagagac

SEQ ID NO: 46 - DNA, 35 bp, synthetic construct
  source 1..35: mol_type = other DNA
gctgaggtaggtgtctttttttttttttttttttt

(19) United States
(12) Patent Application Publication
(10) Pub. No.: US 2023/0250468 A1
(43) Pub. Date: Aug. 10, 2023

(54) HOMOGENOUS ASSAYS IN MICRODROPLETS

(71) Applicant: Scribe Biosciences, Inc., San Francisco, CA (US)

(72) Inventors: Nathan Schoepp, San Francisco, CA (US); Justin Madrigal, San Francisco, CA (US); Maithreyan Srinivasan, San Francisco, CA (US); Russell Cole, San Francisco, CA (US); Codian Powell, San Francisco, CA (US); Adam Whitman, San Francisco, CA (US)

(21) Appl. No.: 18/156,032

(22) Filed: Jan. 18, 2023

Related U.S. Application Data
(60) Provisional application No. 63/300,459, filed on Jan. 18, 2022.

(51) Int. Cl. C12Q 1/6811; B01L 3/00; C12N 9/22

(52) U.S. Cl. CPC C12Q 1/6811; B01L 3/502769; C12N 9/22; C12Y 301/27; C12Q 2600/166

(57) ABSTRACT

Provided herein are compositions, systems, kits, and methods for detecting the presence or absence of a target protein in a discrete entity comprising: a) generating a discrete entity (e.g., microdroplet) comprising: i) a first cell that may secrete, or surface express, a target protein, ii) a quenched oligonucleotide probe, iii) first antibody-oligonucleotide conjugate or a particle-oligonucleotide conjugate, and a second antibody-oligonucleotide conjugate that bind the target protein in proximity to each to form an oligonucleotide template structure (OTS), and a nickase enzyme that cleaves the quenched oligonucleotide probe when it is hybridized to the OTS such that a detectable dye (e.g., fluorescent dye) is released and generates a signal; and b) detecting the presence or absence of the signal from the detectable dye.

The present application claims priority to U.S. Provisional application Serial No. 63/300,459 filed Jan. 18, 2022, which is herein incorporated by reference in its entirety.

### SEQUENCE LISTING

The text of the computer readable sequence listing filed herewith, titled “39801-202_SEQUENCE_LISTING”, created Jan. 18, 2023, having a file size of 75,898 bytes, is hereby incorporated by reference in its entirety.

### FIELD OF THE INVENTION

Provided herein are compositions, systems, kits, and methods for detecting the presence or absence of a target protein in a discrete entity comprising: a) generating a discrete entity (e.g., microdroplet) comprising: i) a first cell that may secrete, or surface express, a target protein, ii) a quenched oligonucleotide probe, iii) first antibody-oligonucleotide conjugate or a particle-oligonucleotide conjugate, and a second antibody-oligonucleotide conjugate that bind the target protein in proximity to each to form an oligonucleotide template structure (OTS), and a nickase enzyme that cleaves the quenched oligonucleotide probe when it is hybridized to the OTS such that a detectable dye (e.g., fluorescent dye) is released and generates a signal; and b) detecting the presence or absence of the signal from the detectable dye.

### BACKGROUND

Immunoassays allow for quantitative measurement of protein targets and are ubiquitous in molecular biology, diagnostics, and drug development. Implementation of multi-step immunoassay protocols in droplets is impossible, which limits the sensitivity that can be achieved in droplets. The vast majority of immunoassays require multiple and sequential reagent addition and washing steps making implementation of these immunoassays in droplets impossible.

### SUMMARY OF THE INVENTION

Provided herein are compositions, systems, kits, and methods for detecting the presence or absence of a target protein in a discrete entity comprising: a) generating a discrete entity (e.g., microdroplet) comprising: i) a first cell that may secrete, or surface express, a target protein, ii) a quenched oligonucleotide probe, iii) first antibody-oligonucleotide conjugate or a particle-oligonucleotide conjugate, and a second antibody-oligonucleotide conjugate that bind the target protein in proximity to each to form an oligonucleotide template structure (OTS), and a nickase enzyme that cleaves the quenched oligonucleotide probe when it is hybridized to the OTS such that a detectable dye (e.g., fluorescent dye) is released and generates a signal; and b) detecting the presence or absence of the signal from the detectable dye.

In some embodiments, provided herein are compositions, kits, and systems comprising: a) a discrete entity (e.g., microdroplet) comprising: i) an oligonucleotide probe comprising: A) a nucleic acid sequence comprising a first sequence and a second sequence; B) a detectable dye (e.g., fluorescent dye); and C) a quencher molecule, wherein the quencher molecule is positioned such that is quenches signal from the detectable dye; ii) a first antibody-oligonucleotide conjugate comprising: a first antibody, or antigen binding fragment thereof (e.g., Fab, Fv, etc.), attached to, or operably linked to, a first oligonucleotide arm which comprises: A) a first region hybridizable (or hybridized) to the first sequence, B) a first template structure forming (TSF) region, and C) first linking region attached to, or operably linked to, the first antibody or antigen binding fragment thereof; iii) a second antibody-oligonucleotide conjugate comprising: a second antibody, or antigen binding fragment thereof, attached to, or operably linked to, a second oligonucleotide arm which comprises: A) a second region hybridizable (or hybridized) to the second sequence, B) a second template structure forming (TSF) region, and C) second linking region attached to, or operably linked to, the second antibody or antigen binding fragment thereof, wherein the first antibody, or antigen fragment thereof, binds a first epitope of a target protein, and the second antibody, or antigen fragment thereof, binds said first epitope or a second epitope of the target protein in proximity to the first epitope such that the first TSF region hybridizes to the second TSF region thereby forming an oligonucleotide template structure (OTS), wherein the OTS allows the oligonucleotide probe to hybridize to both the first and second regions; and iii) a nickase enzyme, wherein the nickase enzyme cleaves the oligonucleotide probe when it is hybridized the OTS such that the detectable dye (e.g., fluorescent dye) is released and is no longer quenched by the quencher molecule (thereby generating a detectable signal from the detectable dye); and b) a carrier fluid, wherein the discrete entity is present in the carrier fluid.

In certain embodiments, provided herein are compositions, kits, and systems comprising: a) a discrete entity comprising: i) an oligonucleotide probe comprising: A) a nucleic acid sequence comprising a first sequence and a second sequence; B) a dye (e.g., fluorescent dye); and C) a quencher molecule, wherein said quencher molecule is positioned such that is quenches signal from said dye; ii) at least one of the following: A) a first antibody-oligonucleotide conjugate comprising: a first antibody or antigen binding fragment thereof, attached to, or operably linked to, a first oligonucleotide arm which comprises: I) a first region hybridizable to said first sequence, II) optionally a first template structure forming (TSF) region, and III) first linking region attached to, or operably linked to said first antibody or antigen binding fragment thereof; B) a particle-oligonucleotide conjugate comprising: I) a particle, II) said first antibody or antigen binding fragment thereof, attached to, or operably linked to said particle, and III) said first oligonucleotide arm which is attached to, operably linked to, said particle; iii) a second antibody-oligonucleotide conjugate comprising: a second antibody or antigen binding fragment thereof, attached to, or operably linked to, a second oligonucleotide arm which comprises: A) a second region hybridizable to said second sequence, B) optionally a second template structure forming (TSF) region, and C) second linking region attached to, or operably linked to, said second antibody or antigen binding fragment thereof, wherein when said first antibody, or antigen fragment thereof, binds a first epitope of a target protein, and said second antibody, or antigen fragment thereof, binds said first epitope or a second epitope of said target protein in proximity to said first epitope this forms an oligonucleotide template structure (OTS), which is stabilized by said first TSF region hybridizing to said second TSF region if both are present; wherein said OTS allows said oligonucleotide probe to hybridize to both said first and second regions; and iii) a nickase enzyme, wherein said nickase enzyme cleaves said oligonucleotide probe when it is hybridized said OTS such that said dye (e.g. fluoresce dye) is released and is no longer quenched by said quencher molecule; and b) a carrier fluid, wherein said discrete entity is present in said carrier fluid.

In particular embodiments, said first oligonucleotide arm further comprises 1 or 2 hinge nucleotides contiguous with said first region that are not hybridized to any nucleotides in said OTS; and/or wherein said a second oligonucleotide arm further comprises 1 or 2 hinge nucleotides contiguous with said second region that are not hybridized to any nucleotides in said OTS. In some embodiments, said particle comprises a bead or nanoparticle that optionally ranges in size from 10 nm - 10 um.

In certain embodiments, provided herein are methods of detecting the presence or absence of a target protein in a discrete entity (e.g., microdroplet) comprising: a) generating a discrete entity in carrier fluid (e.g., combining multiple droplets, or forming an initial droplet), wherein the discrete entity comprises: i) a first cell that may secrete a target protein (e.g., when induced or constitutively); ii) an oligonucleotide probe comprising: A) a nucleic acid sequence comprising a first sequence and a second sequence; B) a detectable dye (e.g., fluorescent dye); and C) a quencher molecule, wherein the quencher molecule is positioned such that is quenches signal from the detectable dye; iii) a first antibody-oligonucleotide conjugate comprising: a first antibody or antigen binding fragment thereof, attached to, or operably linked to, a first oligonucleotide arm which comprises: A) a first region hybridizable (or hybridized) to the first sequence, B) a first template structure forming (TSF) region, and C) first linking region attached to, or operably linked to, the first antibody or antigen binding fragment thereof; iv) a second antibody-oligonucleotide conjugate comprising: a second antibody, or antigen binding fragment thereof, attached to, or operably linked to, a second oligonucleotide arm which comprises: A) a second region hybridizable (or hybridized) to the second sequence, B) a second template structure forming (TSF) region, and C) second linking region attached to, or operably linked to, the second antibody or antigen binding fragment thereof, wherein the first antibody, or antigen fragment thereof, binds a first epitope of the target protein, and the second antibody, or antigen fragment thereof, binds said first epitope or a second epitope of the target protein in proximity to the first epitope such that the first TSF region hybridizes to the second TSF region thereby forming an oligonucleotide template structure (OTS), wherein the OTS allows the oligonucleotide probe to hybridize to both the first and second regions; and v) a nickase enzyme, wherein the nickase enzyme cleaves the oligonucleotide probe when it is hybridized to the OTS (i.e., cleaves the oligonucleotide probe only when it is hybridized to the OTS structure) such that the detectable dye (e.g., fluorescent dye) is released and is no longer quenched by the quencher molecule thereby generating the signal; and b) detecting the presence or absence of the signal from the detectable dye (e.g., to get the secretion or cell-surface expression of the target protein in the discrete entity).

In particular embodiments, provided herein are methods of detecting the presence or absence of a target protein in a discrete entity comprising: a) generating a discrete entity in carrier fluid, wherein said discrete entity comprises: i) a first cell that may secrete a target protein; ii) an oligonucleotide probe comprising: A) a nucleic acid sequence comprising a first sequence and a second sequence; B) a fluorescent dye; and C) a quencher molecule, wherein said quencher molecule is positioned such that is quenches signal from said fluorescent dye; iii) at least one of the following: A) a first antibody-oligonucleotide conjugate comprising: a first antibody or antigen binding fragment thereof, attached to, or operably linked to, a first oligonucleotide arm which comprises: I) a first region hybridizable to said first sequence, II) optionally a first template structure forming (TSF) region, and III) first linking region attached to, or operably linked to, said first antibody or antigen binding fragment thereof; B) a first particle-oligonucleotide conjugate comprising: I) a particle, II) said first antibody or antigen binding fragment thereof, attached to, or operably linked to said particle, and III) said first oligonucleotide arm which is attached to, operably linked to, said particle; iv) a second antibody-oligonucleotide conjugate comprising: a second antibody or antigen binding fragment thereof, attached to, or operably linked to, a second oligonucleotide arm which comprises: A) a second region hybridizable to said second sequence, B) optionally a second template structure forming (TSF) region, and C) second linking region attached to, or operably linked to, said second antibody or antigen binding fragment thereof, wherein said first antibody, or antigen fragment thereof, binds a first epitope of said target protein, and said second antibody, or antigen fragment thereof, binds said first epitope or a second epitope of said target protein in proximity to said first epitope thereby forming an oligonucleotide template structure (OTS), which is stabilized by said first TSF region hybridizing to said second TSF region if both are present; wherein said OTS allows said oligonucleotide probe to hybridize to both said first and second regions; and v) a nickase enzyme, wherein said nickase enzyme cleaves said oligonucleotide probe when it is hybridized to said OTS such that said fluorescent dye is released and is no longer quenched by said quencher molecule thereby generating said signal; and b) detecting the presence or absence of said signal from said fluorescent dye.

In particular embodiments, provided herein are compositions comprising: a) an oligonucleotide probe comprising: A) a nucleic acid sequence comprising a first sequence and a second sequence; B) a dye (e.g., fluorescent dye); and C) a quencher molecule, wherein said quencher molecule is positioned such that is quenches signal from said dye; b) at least one of the following: i) a first antibody-oligonucleotide conjugate comprising: a first antibody or antigen binding fragment thereof, attached to, or operably linked to, a first oligonucleotide arm which comprises: A) a first region hybridizable to said first sequence, B) optionally a first template structure forming (TSF) region, and C) first linking region attached to, or operably linked to said first antibody or antigen binding fragment thereof; ii) a particle-oligonucleotide conjugate comprising: A) a particle, B) said first antibody or antigen binding fragment thereof, attached to, or operably linked to said particle, and C) said first oligonucleotide arm which is attached to, operably linked to, said particle; c) a second antibody-oligonucleotide conjugate comprising: a second antibody or antigen binding fragment thereof, attached to, or operably linked to, a second oligonucleotide arm which comprises: A) a second region hybridizable to said second sequence, B) optionally a second template structure forming (TSF) region, and C) second linking region attached to, or operably linked to, said second antibody or antigen binding fragment thereof, wherein when said first antibody, or antigen fragment thereof, binds a first epitope of a target protein, and said second antibody, or antigen fragment thereof, binds said first epitope or a second epitope of said target protein in proximity to said first epitope this forms an oligonucleotide template structure (OTS), which is stabilized by said first TSF region hybridizing to said second TSF region if both are present; wherein said OTS allows said oligonucleotide probe to hybridize to both said first and second regions; and wherein said first oligonucleotide arm further comprises 2 hinge nucleotides contiguous with said first region that are not hybridized to any nucleotides in said OTS; and/or wherein said a second oligonucleotide arm further comprises 2 hinge nucleotides contiguous with said second region that are not hybridized to any nucleotides in said OTS. In some embodiments, the compositions further comprise said target protein, such that said OTS is formed where said first region is hybridized to said first sequence and said second region is hybridized to said second sequence.

In some embodiments, provided herein are methods of generating a plurality of discrete entities and detecting the presence or absence of a signal therefrom comprising: a) flowing a first dispersed phase fluid in a first inlet channel of a co-flow micro-capillary droplet maker, wherein said first dispersed phase fluid contains particles labelled with an enzyme or a first oligonucleotide, wherein said co-flow micro-capillary droplet maker comprises: i) said first inlet channel, ii) a second inlet channel, iii) a merger region, iv) at least one continuous phase inlet channel, v) a droplet generating region in fluid communication with said at least one continuous phase inlet channel and said merger region, vi) an outlet channel in fluid communication with said droplet generating region; and vii) a signal detecting element in proximity to said outlet channel; b) flowing a continuous phase fluid in said at least one continuous phase inlet channel; and c) flowing a second dispersed phase fluid in said second inlet channel of said co-flow micro-capillary droplet maker such that is merges with said first dispersed phase fluid at said merger region to create a mixed dispersed phase fluid, wherein said mixed dispersed phase fluid flows into said droplet generating region such that a plurality of microdroplets are generated by said droplet generating region which flow in said continuous phase fluid into said outlet channel, wherein said second dispersed fluid contains: i) a substrate that generates a signal when acted upon by said enzyme, or ii) a second oligonucleotide and an enzyme, wherein said second oligonucleotide hybridizes to said first oligonucleotide and comprises a dye and a quencher molecule that is positioned such that is quenches signal from said dye, and wherein said enzyme cleaves said second oligonucleotide when it is hybridized to said first oligonucleotide to generate a signal; and d) detecting the presence or absence of said signal in each of said plurality of microdroplets using said signal detecting element.

In certain embodiments, the particles are selected from cells, beads, or nanoparticles. In other embodiments, the co-flow micro-capillary droplet maker further comprises a sorting element and a sorting channel in fluid communication with said outlet channel, and wherein the method further comprises sorting each of said plurality of microdroplets into either said sorting channel, or staying in said outlet channel, based on either the presence or the absence of said signal in each microdroplet. In particular embodiments, the substrate comprise a fluorogenic substrate. In additional embodiments, the presence of said signal in a particular microdroplet indicates the presence of said first particle in said particular microdroplet.

In certain embodiments, wherein the presence or absence of a second target protein is detected, wherein the method further comprises: c) generating said discrete entity such that it further comprises: i) a second oligonucleotide probe having a second fluorescent dye that is quenched by a second quencher molecule; ii) third antibody-oligonucleotide conjugate or second particle-oligonucleotide conjugate, and fourth antibody-oligonucleotide conjugates that are configured to generate a second OTS that can bind said second oligonucleotide probe; iii) a second nickase enzyme, wherein said second nickase enzyme cleaves said second oligonucleotide probe if said second target protein is present thereby generating a second signal from said second fluorescent dye, and iv) optionally a second cell; and d) detecting the presence or absence of said second signal from said second fluorescent dye.

In particular embodiments, wherein: A) the absence of the signal indicates the target protein is not present in solution in the discrete entity after being secreted by the first cell and/or is not expressed on the surface of the first cell, or B) the presence of the signal indicates the target protein is present in solution in the discrete entity after being secreted by the first cell and/or is expressed on the surface of the first cell. In other embodiments, the methods further comprise: c) flowing the discrete entity in the carrier fluid in a microfluidic fluidic device, and d) sorting the discrete entity into a waste channel if the signal is not detected or into a keep channel if the signal is detected. In some embodiments, the sorting is performed without any visual imaging.

In particular embodiments, the presence or absence of a second target protein is detected, wherein the method further comprises: c) generating the discrete entity such that it further comprises: i) a second oligonucleotide probe having a second fluorescent dye that is quenched by a second quencher molecule; ii) third and fourth antibody-oligonucleotide conjugates that are configured to generate a second OTS that can bind the second oligonucleotide probe; iii) a second nickase enzyme, wherein the second nickase enzyme cleaves the second oligonucleotide probe if the second target protein is present thereby generating a second signal from the second fluorescent dye, and iv) optionally a second cell; and d) detecting the presence or absence of the second signal from the second fluorescent dye. In further embodiments, wherein: A) the absence of the second signal indicates the second target protein is not present in solution in the discrete entity after being secreted by the first or second cell and/or is not expressed on the surface of the first or second cell, or B) the presence of the second signal indicates the second target protein is present in solution in the discrete entity after being secreted by the first or second cell and/or is expressed on the surface of the first or second cell.

In other embodiments, the discrete entity further comprises cell media. In certain embodiments, the first cell is stained with a first detectable cell dye. In some embodiments, the first cell is a Car-T cell or a hybridoma. In additional embodiments, the first cell secretes, or can be induced to secrete, the target protein. In additional embodiments, the discrete entity further comprises a second cell, and optionally, wherein the second cell is stained with a second detectable dye. In further embodiments, the first cell is a target cell, and the second cell is an effector cell, and wherein the first and second cells interact to cause secretion of the target protein from the first or second cell. In other embodiments, there are no other cells present in the combined discrete entity besides the first and second cells.

In certain embodiments, there are no other cells present in the discrete entity except the first cell. In other embodiments, the discrete entity further comprises the target protein. In additional embodiments, the target protein comprises a cytokine. In additional embodiments, the cytokine is IFN-γ, TNF-α, GM-CSF, and/or IL-2. In other embodiments, the target protein comprises a third antibody secreted by the first cell, which is a hybridoma.

In certain embodiments, the oligonucleotide probe comprises a molecular beacon probe. In other embodiments, the first and/or second cell are each independently selected from the group consisting of: an antigen presenting cell (APC), a T-cell, a B-cell, a myeloma, a cancer cell, an endothelial cell, and an epithelial cell. In some embodiments, the first and/or second cells are human cells. In certain embodiments, the first and second antibody, or antigen binding fragments thereof, i) both bind the first epitope, and/or ii) are different; or iii) are the same.

In particular embodiments, the discrete entity is a droplet. In further embodiments, the droplet comprises an aqueous fluid which is immiscible in the carrier fluid. In additional embodiments, the discrete entity has a diameter of from about 1 µm to 1000 µm. In further embodiments, the discrete entity has a volume of from about 1 femtoliter to about 1000 nanoliters, or from 10 to 800 picoliters. In other embodiments, the carrier fluid comprises oil, and wherein the discrete entity further comprises an aqueous solution. In some embodiments, the nickase is selected from the group consisting of: Nt.BsmAI, Nb.BsrDI, Nb.BbvCI, Nt.BspQI, Nt.CviPII, Nt.BstNBI, Nt.AlwI, Nt.BbvCI, Nb.BsmI, Nb.BssSI, and Nb.BtsI.

In certain embodiments, the discrete entity flows in the carrier fluid in a microfluidic device or is generated by combining two or more discrete entities in a microfluidic device. In particular embodiments, the microfluidic device comprises: i) an inlet channel, ii) a sorting channel in fluid communication with the inlet channel, iii) first and second outlet channels in fluid communication with the sorting channel, wherein the first outlet channel comprises a merger region, iv) a sorting element positioned in proximity to the sorting channel, and v) a trapping element positioned in proximity to the merger region. In some embodiments, the discrete entity is trapped at the merger region and merged with a second discrete entity (e.g., containing reagents or cell(s), that may induce the first cell).

In some embodiments, the sorting element comprises a first sorting electrode that exerts an electromagnetic force sufficient to sort a discrete entity in the sorting channel to the first outlet channel. In certain embodiments, the electromagnetic force is a dielectrophoretic force or an electrophoretic force. In other embodiments, the trapping element exerts an electromagnetic force, exerts a mechanical force, or a combination thereof sufficient to trap multiple discrete entities in the discrete entity merger region for a time sufficient for the discrete entities to combine to form a combined discrete entity. In other embodiments, the trapping element comprises a first trapping electrode that exerts an electromagnetic force sufficient to trap discrete entities in the merger region for a time sufficient for discrete entities to combine to form the combined discrete entity. In other embodiments, the electromagnetic force is a dielectrophoretic force or an electrophoretic force.

In additional embodiments, the discrete entity has a diameter of from about 1 µm to 1000 µm (e.g., 1 ... 100 ... 300 ... 700 ... 1000 µm). In other embodiments, the combined discrete entity has a volume of from about 1 femtoliter to about 1000 nanoliters, or from 10 to 800 picoliters (e.g., 1 femtoliter .... 10 nanoliters ... 1000 nanoliters).

### DESCRIPTION OF THE FIGURES

The patent or application file contains at least one drawing executed in color. Copies of this patent or patent application publication with color drawings will be provided by the Office upon request and payment of the necessary fee.

FIG. 1A shows first and second exemplary antibody-oligonucleotides and how they bind in proximity to each other on different epitopes of a target protein such that a template structure is formed. An oligonucleotide probe hybridizes to the template structure, which then allows the nickase enzyme to cleave the probe releasing the fluorophore and quencher. In this regard, the signal from the fluorophore is no longer quenched and is detectable, indicating that the target protein has been detected. This cycle repeats as more and more oligonucleotide probes are cleaved.

FIG. 1B shows certain parts of the oligo template structure labeled, along with a nickase enzyme and an oligonucleotide probe.

FIG. 2A shows an exemplary assay workflow. First, a droplet is generated that has first and second antibody-oligonucleotide structures, a quenched oligonucleotide probe, a nickase enzyme, and a cell (a CAR-T cell is shown in this figure as an example). Next, the cell secretes a target protein (cytokines are shown as an example), which prompts the formation of the template structure as each antibody binds a different epitope on the target protein. The quenched oligonucleotide probe (having a quenched detectable label and quencher) hybridizes to the template structure. This forms a cleavage structure for the nickase which cleaves the probe, liberating the detectable label (e.g., fluorophore) from being quenched by the quencher. This process repeats, generating more and more signal. Signal from the non-quenched detectable labels is detected, indicating the presence of the target protein in the droplet.

FIG. 2B shows a similar assay as FIG. 2A, but the cell is a hybridoma that secretes a monoclonal antibody as the target protein.

FIG. 2C also shows a similar assay as FIG. 2A, but further includes a target or effector cell (e.g., antigen presenting cell), which interacts with the other cell (e.g., CAR-T cell) to cause expression of a target protein from the CAR-T cell.

FIG. 3 provides a block schematic diagram of an exemplary microfluidic device having an inlet channel, a sorting channel, a sorting element, first and second outlet channels, a trapping element, a discrete entity merger region, and upstream and a downstream regions.

FIG. 4 provides an image of an exemplary microfluidic device having a spacer fluid channel, a bias fluid channel, a laminating oil inlet channel, a concentric sorter channel, a flow divider, and a recess according to embodiments of the present disclosure.

FIG. 5 provides images of an exemplary microfluidic device having a concentric sorter channel, a recess, and an approximately triangular downstream region according to embodiments of the present disclosure.

FIG. 6, panels i-iv, show a zoomed-out view of an exemplary integrated droplet sorter-combiner. A droplet (e.g., containing a first cell, the template structure forming reagents herein, oligonucleotide probe, and nickase enzyme) is detected as it enters the droplet sorting region (i), the sorting electrode is actuated to redirect the drop towards the assembly lane (ii), and the sorted droplet merges with the droplet-in-assembly at the DEP trap to form a combined droplet (iii) (e.g., containing a second cell). Following assembly, the DEP trap is turned off to release the combined droplet (iv). Panels v-viii show a close-up of the merging process. Four droplets (e.g., containing a total of two cells) are sorted by their fluorescent signature and directed to the DEP trap for merging (v). As the droplets encounter the actuated trap, they are sequentially merged into the assembled droplet (vi-vii). The electrode is then temporarily turned off so the assembled droplet may be released and recovered downstream (viii) (e.g., after being sorted for the presence of live cells).

FIG. 7 provides a schematic flow diagram of a method of selectively combining discrete entities using a microfluidic device according to embodiments of the present disclosure.

FIG. 8 provides a schematic showing example configurations for trapping a discrete entity (e.g., droplet). Panel i) shows a bipolar electrode pair embedded in the same side wall of a channel. Panel ii) shows a bipolar electrode pair embedded on opposite sides of channel. Panel iii) shows bipolar electrode pair embedded in the floor or ceiling of a channel.

FIG. 9 provides a schematic showing exemplary configurations for directing discrete entities to a discrete entity merger region. Panel i) shows application of a lamination flow to confine the laminar flow containing the droplet to the side wall of the channel. Panel ii) shows a partial height flow divider that allows fluid, but not droplets to enter the center portion of the channel. Panel iii) shows a configuration where a groove of similar height to the droplet dimensions is patterned near the side wall of a channel, while the rest of the channel is constructed with a reduced height to exclude droplets. Panel iv) shows a porous flow divider that allows fluid, but not droplets to enter the center portion of the channel. Panel v) shows a partial height flow dividers that direct droplets to a trap at the center of the microfluidic channel.

FIG. 10 provides a schematic showing an exemplary embodiment wherein trapping is facilitated by a mechanical valve. Panel i) shows an initial stage where the discrete entities are trapped by the valve. Panel ii) shows a second stage wherein the discrete entities have been combined, e.g. due to electrical, chemical, or other means. Panel iii) shows a third stage where the combined discrete entity is released by opening the valve and carried downstream.

FIG. 11 provides a schematic showing exemplary embodiments with different channel geometries in proximity to an electromagnetic trapping element. Panel i) shows a discrete entity merger region upstream of a bend in the channel wall. Panel ii) shows a discrete entity merger region in a lateral facet in the channel wall. Panel iii) shows a discrete entity being trapped in a region that is vertically taller than the main channel.

FIG. 12 shows signal amplification using four different nickase enzymes (Nt.BsmAI, Panel A; Nb.BsrDI, Panel B; Nb.BbvCI, Panel C; and Nb.BtsI, Panel D) with hairpin proxy templates as described in Example 1.

FIG. 13 shows an antibody-oligo template structure vs. hairpin proxy used in Example 2 below.

FIG. 14 shows enzyme compatibility in media (XVIVO) for Nt.BsmAI (A) and Nb.Btsl (B) as described in Example 2.

FIG. 15 shows antibody-oligo template structure vs streptavidin/biotinylated oligos proxy as described in Example 3.

Example 16 shows detection of streptavidin in droplets using biotinylated oligos.

FIG. 17 shows the location of the hinge region nucleotides in the template arms and probe.

FIG. 18 shows the results of Example 4, which shows average signal amplification rate over the first 20 min of the reaction can vary based on having adenines or thymines at the hinge positions in the probe or in the template arms of the oligo-antibody or oligo-particle constructs.

FIG. 19 shows the location of the stem region in the proxy template used in Example 5.

FIG. 20 shows results from Example 5 where the proxy templates (with 0, 1, 2, 3, 4, 5, or 6 stem nucleotides) were combined with probe, nickase, and Pluronic F-68 in rCutSmart buffer (NEB) and incubated at 37° C. Fluorescence was measured every two minutes over three hours. Results shows real time signal amplification curves for each sample.

FIG. 21 shows the structure of various templates used in Example 6 and how they bind the probe.

FIG. 22 shows results from Example 6 where the various structures from FIG. 21 were combined with probe, nickase, and Pluronic F-68 in rCutSmart buffer (NEB) and incubated at 37° C. Fluorescence was measured every two minutes over three hours. Results show real time signal amplification curves for each sample.

FIG. 23 shows the results of Example 7 where it was shown that higher enzyme concentrations partially overcome the inhibitory effect of high salt concentrations in the assay buffer.

FIG. 24 shows results of Example 8, wherein it was shown monoclonal and polyclonal antibodies can be used with the method described therein.

FIG. 25 shows results of Example 9 that shows that 1, 5, and 10 nM IFN-γ can be detected in droplets using the homogeneous assay with antibody-oligo reagents.

FIG. 26 shows results of Example 10 that show that the assay can detect 10 nM IFN-γ even when a cell is present in the droplet.

FIG. 27 shows an assay format described in Example 11 where the proximity assay occurs on the surface of a bead or nanoparticle.

FIG. 28A shows various reagents of the co-flow droplet generation described in Example 12.

FIG. 28B shows an exemplary co-flow droplet generation device, with exemplary components flowing therein as described in Example 12, which allows one to determine if a particular droplet contains a cell, particle, or bead labelled with the enzyme.

FIG. 29 shows plate reader data from Example 13 for two different potential enzymes that can be used in the microdroplet assays, including rCutSmart (FIG. 29, left graph) and beta-galactosidase (FIG. 29, right graph).

FIG. 30 shows an image of beads generating a homogeneous signal in droplets, labelled as described in Example 14.

FIG. 31 shows the results of Example 15 where the labelling of cells with β-galactosidase was described, such that droplets that contain a cell can be detected based on the homogeneous signal generated by the β-galactosidase-labelled cell. FIG. 31 shows labelled cells generating a homogeneous signal in droplets. Cells are denoted with red arrows.

### DETAILED DESCRIPTION

Provided herein are compositions, systems, kits, and methods for detecting the presence or absence of a target protein in a discrete entity comprising: a) generating a discrete entity (e.g., microdroplet) comprising: i) a first cell that may secrete, or surface express, a target protein, ii) a quenched oligonucleotide probe, iii) first antibody-oligonucleotide conjugate or a particle-oligonucleotide conjugate, and a second antibody-oligonucleotide conjugate that bind the target protein in proximity to each to form an oligonucleotide template structure (OTS), and a nickase enzyme that cleaves the quenched oligonucleotide probe when it is hybridized to the OTS such that a detectable dye (e.g., fluorescent dye) is released and generates a signal; and b) detecting the presence or absence of the signal from the detectable dye.

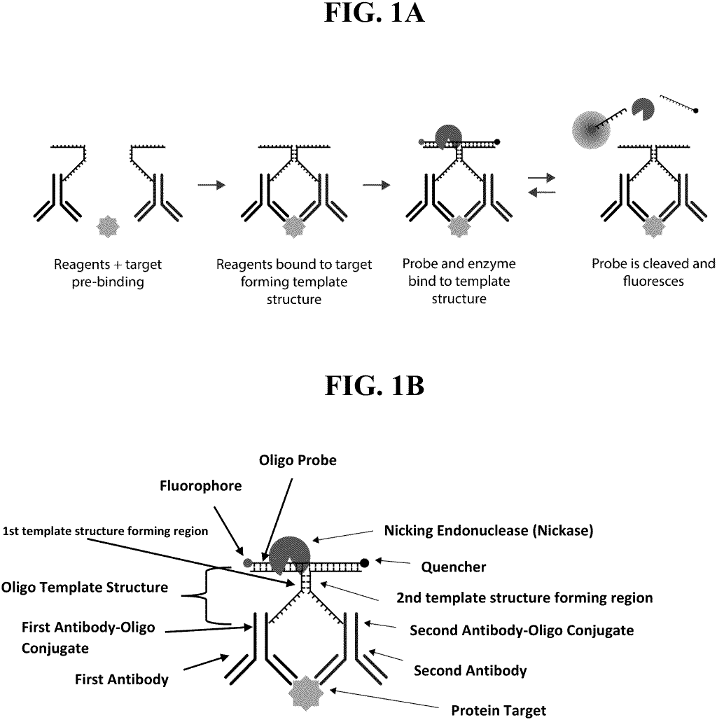

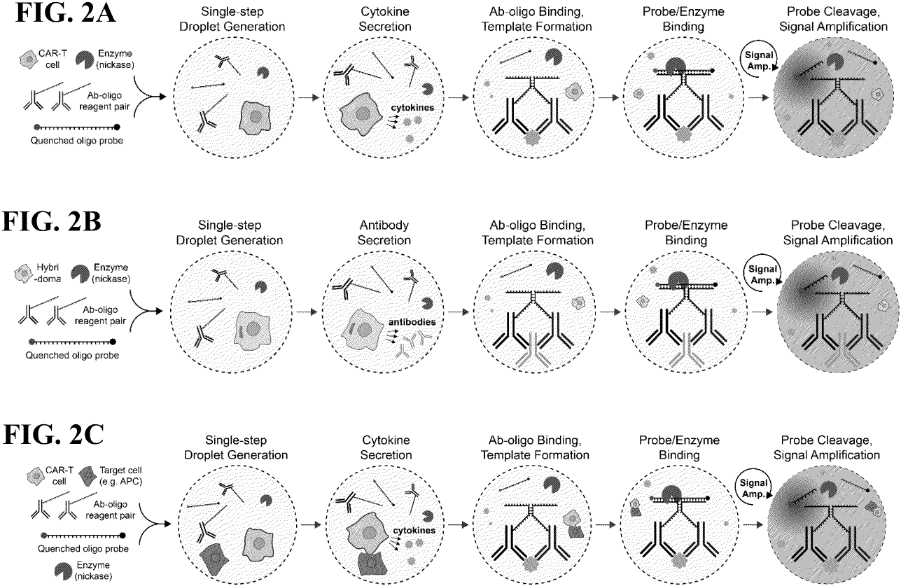

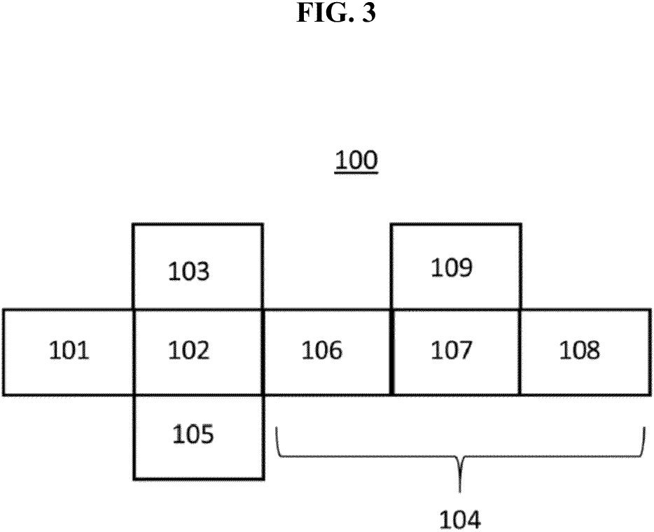

In certain embodiments, methods and compositions generally employ oligonucleotide template structure forming reagents, and associated reagents, as shown in FIGS. 1 and 2, and as shown in Deng et al., Anal Chem 86(14): 7009-7016 (see, e.g., scheme 1), which is herein incorporated by reference in its entirety. In certain embodiments, such assay reagents (for including in the discrete entities herein), are as follows. Two antibodies specific to different epitopes of the target protein are each labelled with a specific oligonucleotide (oligo). These oligos form the two halves of a template structure that when formed allows for binding of an oligo probe. The template structure will only form when both antibodies are bound to the target protein and in close proximity to one another. The probe is labelled with a fluorophore of choice and a quencher and is not fluorescent in its un-cleaved state. When the probe is bound to the oligo template structure a nickase enzyme cleaves the probe only (leaving the template structure intact). The cleaved probe dissociates from the template structure and fluoresces since it is no longer quenched. In certain embodiments, all reagents (e.g., the two Ab-oligo conjugates, the probe, and the nickase) are loaded into the assay (in bulk format or droplets) at the start of the assay. In other embodiments, multiple discrete entities are combined to bring together all the assay reagents. No wash steps are required and the signal is amplified via the linear cleavage reaction of the probe. In certain embodiments, regents are employed in a discrete entity to form two, three, or more template structures, along with multiple different nickase enzymes and different oligonucleotide probes (e.g., that provide different colors). This regard, multiple different target proteins can be detected. In certain embodiments, the oligonucleotide arms attached to the antibodies (or antibody fragments) are attached directly to such antibodies/fragments, rather than having a linker such as streptavidin linkage), which allows for a more sensitive limit of detection by reducing background from the two oligos both binding to the same streptavidin or similar linkage.

In certain embodiments, the first and second antibodies (or antigen binding fragments thereof) bind the same epitope on the target protein (e.g., even though the two antibodies or fragments are different molecules). In other embodiments, such as in the case of multimeric proteins (e.g., dimers, trimers, etc.) the same antibody can bind more than one of the same epitope on the protein, so the first and second antibodies (or antigen binding fragments thereof) are the same molecule (e.g., same monoclonal antibody).

In some embodiments, the discrete entities herein are flowed in a microfluidic device, which may be used to combine multiple drops such that all the reagents are combined into a single discrete entity. In certain embodiments, the microfluidic devices employed herein comprise a microenvironment on Demand (MOD) device, described in PCT application WO2020232072A1 and Cole et al., Proc. Natl. Acad. Sci., 114(33): 8728-8733, 2017, which are both incorporated by reference herein in their entireties. In certain embodiments, the MOD platform is composed of a combination of deterministic single-cell droplet sorter and droplet-assembler that can selectively assemble cells and reagents. MOD performs a cyclic buildup and release of designer droplets through the merging of select droplets on a defined dielectrophoretic trapping position inside the microfluidic device (FIG. 6). This approach is advantageous because it is less prone to contamination, higher throughput, and requires fewer moving parts than other devices. The flexible nature of the MOD platform makes it a well-suited technology to perform integrated and functional cell-cell, cell-ECM interaction analysis and link any perturbations to select expressed gene sequences or transcriptome profiles at a single cell level. Essentially, MOD allows for precise, flexible, scalable liquid handling that can build a large number of predetermined reaction conditions.

MOD not only allows for the sorting and combination of particulates (e.g., cells, reagents for making oligo template structures, nickases, quenched oligonucleotide probes, etc.), but also sorts and assembles diverse droplet contents. Furthermore, droplet experiments constructed with MOD are compartmentalized and miniaturized (e.g., ~100 pL) providing contained reactions in concentrated volumes. These two aspects of MOD, reagent selection and reaction miniaturization, provide a powerful approach to phenotypically screen large numbers of single cells.

In certain embodiments, the MOD platform is employed and droplet manipulation and sorting is achieved by electrowetting, the modification of the wetting properties of a surface with an applied electric field. Electrowetting manipulation of droplets in a microfluidic device may be achieved through the application of differential voltages to different regions in an electrode grid (see, U.S. Pat. 6,911,132, herein incorporated by reference). Alternatively, droplet actuation and sorting can be achieved using opto-electrowetting, where localized electric fields are triggered through the selective application of light to a photoconductive layer (see, U.S. Pat. 6,958,132, which is herein incorporated by reference in its entirety).

In certain embodiments, droplet-based cell culture is performed using porous materials. The duration of cell culture in sub-nanoliter droplets is limited by a finite amount of encapsulated media and localized buildup of metabolic waste products. In cases where longer duration incubations are desired or required, it may be appropriate to convert a droplet to a media-permeable format while keeping encapsulated objects in place. This can be achieved by flowing hydrogel precursors into droplets along with cells, then triggering gelation to form either gel beads or permeable capsules. After gelation, the emulsion is broken, the emulsion oil is removed, and the cell-laden (e.g., hybridoma-laden, target cell, etc.) beads or capsules are suspended in media and cultured for a time. Examples of the hydrogel bead approach are given in Wan et al., (Polymers (Basel)., vol. 4, no. 2, pp. 1084-1108, 2012), Utech et al., (Adv. Healthc. Mater., 2015), and Dolega et al. (Biomaterials, vol. 52, no. 1, pp. 347-357, 2015.) - all of which are herein incorporated by reference in their entireties. Examples of permeable capsules are given by Yu et al, (Biomed. Microdevices, vol. 17, no. 2, 2015.), van Loo et al (Mater. Today Bio, vol. 6, no. February, p. 100047, 2020.), and Leonaviciene et al. (Lab Chip, no. Advanced Article, 2020), all of which are herein incorporated by reference in their entireties. Extended cell culture (e.g., after a target protein detection assay as described herein) is especially useful in cases where cell proliferation is important, such as clonal expansion of single cells and cell-cell interaction assays where proliferation is a readout. In some cases, it may be necessary to break down a gel bead or capsule via chemical, enzymatic, or thermal means in order to access the contents for further processing.

Discrete entities (e.g., droplets) as used or generated in connection with the subject methods, devices, and/or systems may be sphere shaped or they may have any other suitable shape, e.g., an ovular or oblong shape. Discrete entities may be droplets. Discrete entities as described herein may include a liquid phase and/or a solid phase material. In some embodiments, discrete entities according to the present disclosure include a gel material. In certain embodiments, the discrete entities comprise double emulsions (or multiple emulsion) or hydrogel shells.

Exemplary double and multiple emulsions are described in U.S. Pat. 9,238,206, which is herein incorporated by reference in its entirety, particularly for such double and multiple emulsions. In general a multiple emulsion describes larger droplets that contain one or more smaller droplets therein. In a double emulsion, the larger droplets may, in turn, be contained within another fluid, which may be the same or different than the fluid within the smaller droplet. In certain embodiments, larger degrees of nesting within the multiple emulsion are possible. For example, an emulsion may contain droplets containing smaller droplets therein, where at least some of the smaller droplets contain even smaller droplets therein, etc. Multiple emulsions can be useful for encapsulating species such as pharmaceutical agents, cells, antibodies, proteins, chemicals, or the like. In certain embodiments, a double emulsion is produced, i.e., a carrying fluid, containing a second fluidic droplet, which in turn contains a first fluidic droplet therein. In some cases, the carrying fluid and the first fluid may be the same. The fluids may be of varying miscibilities, e.g., due to differences in hydrophobicity. For example, the first fluid may be water soluble, the second fluid oil soluble, and the carrying fluid water soluble. This arrangement is often referred to as a w/o/w multiple emulsion (“water/oil/water”). Another double emulsion may include a first fluid that is oil soluble, a second fluid that is water soluble, and a carrying fluid that is oil soluble. This type of double emulsion is often referred to as an o/w/o double emulsion (“oil/water/oil”). It should be noted that the term “oil” in the above terminology merely refers to a fluid that is generally more hydrophobic and not miscible in water, as is known in the art. Thus, the oil may be a hydrocarbon in some embodiments, but in other embodiments, the oil may comprise other hydrophobic fluids.

In certain embodiments, the discrete entities herein comprise a hydrogel shell or microcapsule, such as exemplified in U.S. Pat. 10,710,045 and U.S. Pat. Pub. 20140127290, both of which are herein incorporated by reference in their entireties, particularly for such hydrogel shells or microcapsules. In certain embodiments, the hydrogel shells for the discrete entities, or microcapsules, comprise a liquid core, and at least one external envelope totally encapsulating the liquid core at its periphery, said external envelope being able to retain the liquid core when the capsule is immersed into a gas and comprising at least one gelled polyelectrolyte and/or a stiffened biopolymer. In certain embodiments, such microcapsules contain a cell and/or other reagents discussed herein. In certain embodiments, a microcapsule refers to a particle or capsule having a mean diameter of about 50 µm to about 1000 µm, formed of a cross-linked hydrogel shell surrounding a biocompatible matrix. The microcapsule may have any shape suitable for cell encapsulation. The microcapsule may contain one or more cells dispersed in the biocompatible matrix, cross-linked hydrogel, or combination thereof, thereby “encapsulating” the cells.

In some embodiments, the subject discrete entities have a dimension, e.g., a diameter, of or about 1.0 µm to 1000 µm, inclusive, such as 1.0 µm to 750 µm, 1.0 µm to 500 µm, 1.0 µm to 100 µm, 1.0 µm to 10 µm, or 1.0 µm to 5 µm, inclusive. In some embodiments, discrete entities as described herein have a dimension, e.g., diameter, of or about 1.0 µm to 5 µm, 5 µm to 10 µm, 10 µm to 100 µm, 100 µm to 500 µm, 500 µm to 750 µm, or 750 µm to 1000 µm, inclusive. Furthermore, in some embodiments, discrete entities as described herein have a volume ranging from about 1 fL to 1 nL, inclusive, such as from 1 fL to 100 pL, 1 fL to 10 pL, 1 fL to 1 pL, 1 fL to 100 fL, or 1 fL to 10 fL, inclusive. In some embodiments, discrete entities as described herein have a volume of 1 fL to 10 fL, 10 fL to 100 fL, 100 fL to 1 pL, 1 pL to 10 pL, 10 pL to 100 pL or 100 pL to 1 nL, inclusive. In addition, discrete entities as described herein may have a size and/or shape such that they may be produced in, on, or by a microfluidic device and/or flowed from or applied by a microfluidic device.

In some embodiments, the discrete entities as described herein are droplets. The terms “drop,” “droplet,” and “microdroplet” are used interchangeably herein, to refer to small, generally spherically structures, containing at least a first fluid phase, such as an aqueous phase (e.g., water), bounded by a second fluid phase (e.g., oil) which is immiscible with the first fluid phase. In some embodiments, droplets according to the present disclosure may contain a first fluid phase (e.g., oil) bounded by a second immiscible fluid phase (e.g., an aqueous phase fluid, such as water). In some embodiments, the second fluid phase is an immiscible phase carrier fluid. Thus, droplets according to the present disclosure may be provided as aqueous-in-oil emulsions or oil in aqueous emulsions. Droplets may be sized and/or shaped as described herein for discrete entities. For example, droplets according to the present disclosure generally range from 1 µm to 1000 µm, inclusive, in diameter. Droplets according to the present disclosure may be used to encapsulate cells, e.g., cells, reagents for making oligo template structures, nickases, quenched oligonucleotide probes, nucleic acids (e.g., DNA), enzymes, reporter dyes, reagents, and a variety of other components. The term droplet may be used to refer to a droplet produced in, on, or by a microfluidic device and/or flowed from or applied by a microfluidic device.

As used herein, the term “dielectrophoretic force” refers to the force exerted on an uncharged particle caused by the polarization of the particle by and interaction with a nonuniform electric field. A dielectrophoretic force can be directed towards (i.e. “attractive dielectrophoretic force”), away from (i.e. “repulsive dielectrophoretic force,”) or in any direction relative to the source of the electric field. Before being contacted by the electric field, the particle can be positively charged, negatively charged, or neutral.

As used herein, the term “electrophoretic force” refers to the force exerted on a charged particle caused by interaction with an electric field. An electrophoretic force can be directed towards (i.e. “attractive electrophoretic force”) away from (i.e. “repulsive electrophoretic force,”) or in any direction relative to the source of the electric field. Before being contacted by the electric field, the particle can be positively charged, negatively charged, or neutral.

As used herein, the term “carrier fluid” refers to a fluid configured or selected to contain one or more discrete entities (e.g., droplets) as described herein. A carrier fluid may include one or more substances and may have one or more properties (e.g., viscosity), which allow it to be flowed through a microfluidic device or a portion thereof. In some embodiments, carrier fluids include, for example: oil or water, and may be in a liquid or gas phase.

The present disclosure provides methods of selectively moving and/or combining discrete entities using the MOD platform (e.g., to combine target and effector cells). FIG. 3 presents a non-limiting, simplified, schematic representation of one type of device and method according to the present disclosure. The microfluidic device of FIG. 3 is labeled as microfluidic device 100. FIG. 3 shows a representation of an inlet channel 101, wherein a discrete entity that is insoluble and/or immiscible in a carrier fluid a carrier fluid can be flowed through the inlet channel 101 to a sorter channel 102 that is in direct fluid communication with inlet channel 101. Next, the discrete entity can be sorted into a first outlet channel 104 or a second outlet channel 105, which are both in direct fluid communication with the sorter channel, by sorting element 103. Sorting element 103 can be, in some cases, an electrode, such as an electrode that is configured to exert a dielectrophoretic force on the discrete entity. Sorting element 103 in FIG. 3 is configured to sort a discrete entity in sorting channel 102 to first outlet channel 104 or second outlet channel 105. In some cases, if the discrete entity is sorted to second outlet channel 105, the discrete entity is sorted to a waste container or is recycled back to inlet channel 101. FIG. 3 shows an embodiment wherein first outlet channel 104 includes an upstream region 106, a discrete entity merger region 107, and a downstream region 108. In some cases, the discrete entity merger region comprises a change in a dimension of the first outlet channel, such as where the discrete entity merger region 107 has a larger cross-sectional area than the upstream region 106.

In addition, the FIG. 3 device includes trapping element 109. In some cases, trapping element 109 includes a trapping electrode, and the trapping electrode is configured to exert a force (e.g. a dielectrophoretic force), that traps the discrete entity in the discrete entity merger region 107. Furthermore, the discrete entity merger region 107 and the trapping element 109 are configured such that a force applied by the trapping electrode in the discrete entity merger region is sufficient to trap a plurality of discrete entities in the discrete entity merger region for a time sufficient for the plurality of discrete entities to combine to form a combined discrete entity (e.g., containing a two cells). In some cases, a trapping electrode is configured to provide an electric field that affects the surface of the discrete entities such that the discrete entities can more easily merge (e.g. the discrete entities will spontaneously merge). In some cases, the affecting is destabilizing.

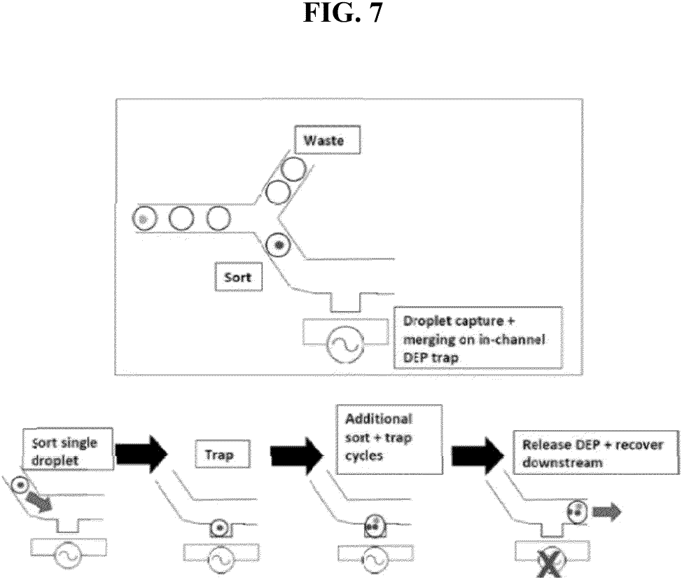

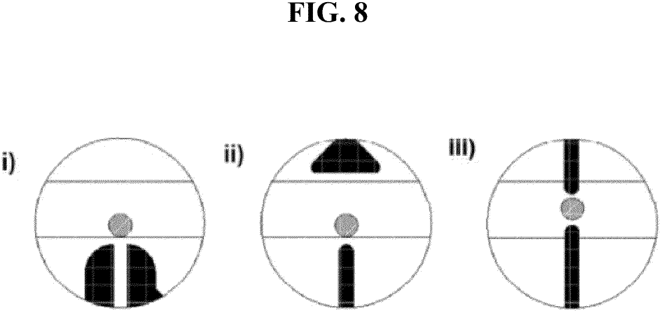

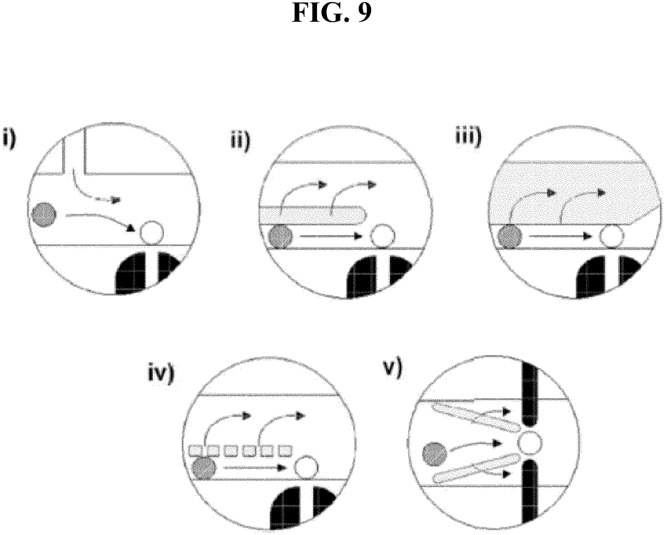

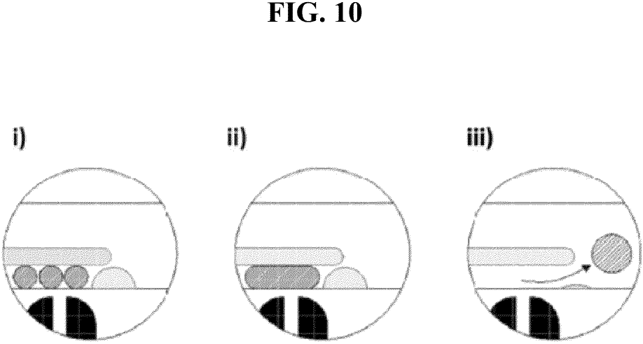

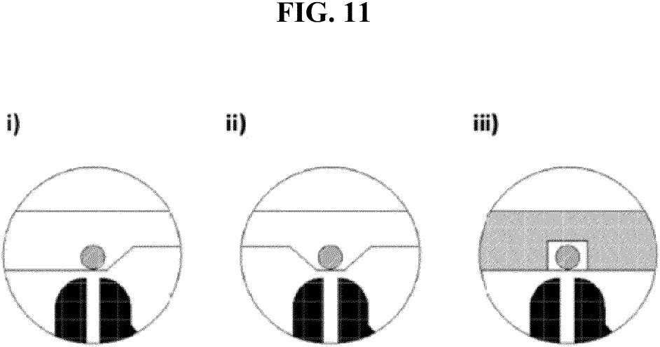

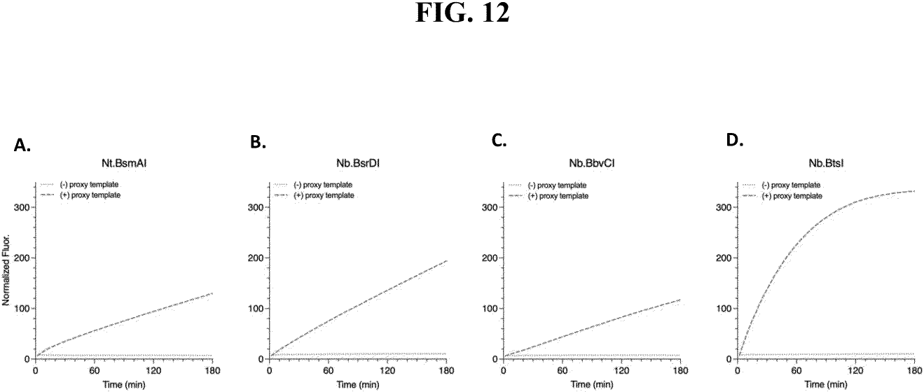

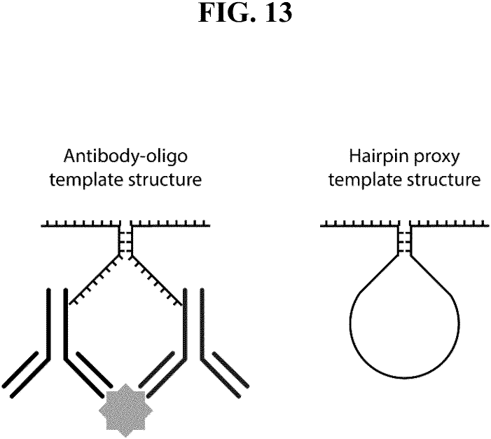

Methods of using the FIG. 3 device include flowing a plurality of discrete entities through inlet channel 101 to sorting channel 102, sorting with sorting element 103 the plurality of discrete entities into first outlet channel 104 or second outlet channel 105, trapping with trapping element 109 at least two or three discrete entities (e.g., one with a target cell and one with an effector cell) in discrete entity merger region 107 for a time sufficient for the discrete entities to combine to form a combined discrete entity. FIG. 7 shows a schematic representation of an exemplary method wherein discrete entities containing cells (e.g., target and effector cells) are selectively combined.

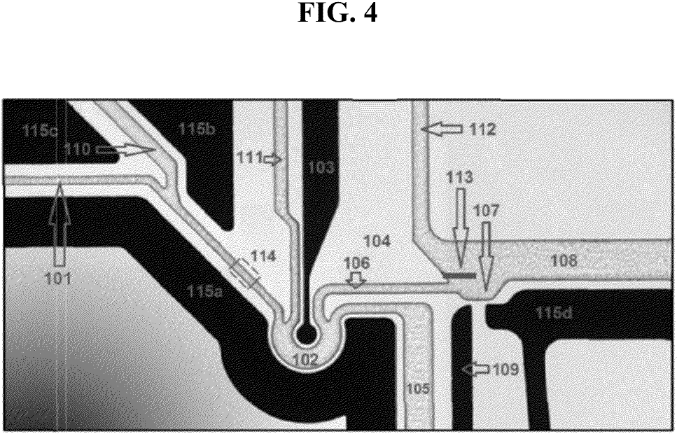

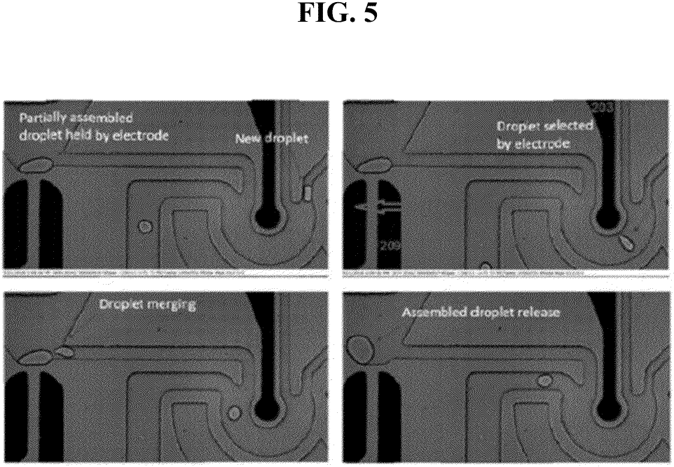

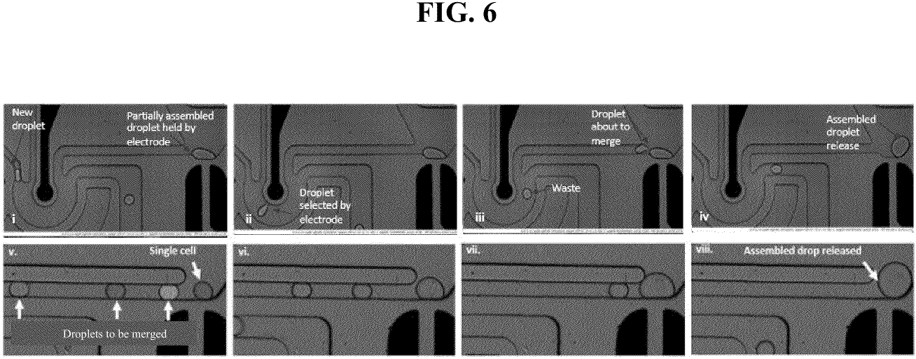

FIG. 4 presents an additional, non-limiting, simplified, schematic representation of one type of a device and method according to the present disclosure. In some cases, the discrete entity merger region includes a recess, such as shown as recess 107 in FIG. 4. In some cases, the discrete entity merger region includes a flow divider, such as shown as flow divider 113 in FIG. 4. In some cases, the device further includes a laminating oil inlet, such as shown as laminating oil inlet 112 in FIG. 4. In some cases, the trapping element includes two electrodes that have a significantly different shape from one another, such as shown as electrodes 109 in FIG. 4. In some cases, the trapping element includes two electrodes that produce a region of high electric field gradients that extends into the microfluidic channel. In some cases, the discrete entity merger region includes a change in the angle of flow between an adjacent upstream region and the discrete entity merger region, e.g. as shown in FIG. 5. In some cases, the device further includes a spacer fluid inlet. As an example, the device in FIG. 4 includes spacer fluid channel 110 in fluid communication with the inlet channel 101. The spacer fluid channel can be configured such that flowing spacer fluid through the spacer fluid channel causes spacer fluid to be located between two discrete entities flowing through the inlet channel, thereby maintaining or increasing the distance between the two discrete entities, thereby allowing each of the two discrete entities to be independently sorted or not sorted.

In some cases, the device further includes a bias fluid inlet. As an example, the device in FIG. 4 includes bias fluid channel 111 in fluid communication with sorter channel 102. The bias fluid channel can be configured such that flowing bias fluid through the bias fluid channel will cause a discrete entity to move closer to a second side wall of the sorter channel and farther away from a first side wall of the sorter channel. Thus, as an example, the spacer fluid inlet 111 would cause the discrete entity to move closer to the wall of the inlet channel that is closer to the bottom of the figure, and further away from the wall closer to the top of the figure. As such, one or more bias fluid channels can be configured such that a discrete entity will preferentially flow to a first outlet location or a second outlet location in the absence of a force from a sorting element. In some cases, the bias fluid inlet channel can be configured such that a discrete entity will preferentially flow to a second outlet channel in the absence of a dielectrophoretic force from a sorting electrode. As an example, the bias fluid inlet 111 in FIG. 4 causes a discrete entity to preferentially flow to second outlet channel 105 in the absence of a force exerted on the discrete entity by the sorting electrodes 103.

In some cases, the device includes a detector configured to detect a discrete entity in the input channel (e.g., to detect if it contain a target protein), wherein the microfluidic device is configured to sort a discrete entity in the sorting channel based on the detection by the detector. As an example, FIG. 4 shows an embodiment in which a discrete entity in detection region 114 of inlet channel 101 can be detected by a detector, after which sorting electrodes 103 can sort the discrete entity into the first outlet channel 104 or the second outlet channel 105. The FIG. 4 devices also includes shielding electrodes 115a, 115b, 115c, and 115d. As used herein, the term “shielding electrode” is used interchangeably with “moat electrode.” Each shielding electrode can be configured to perform one or more functions including: at least partially shielding discrete entities from undesired electromagnetic fields, assisting with the sorting of discrete entities, and assisting with the trapping of discrete entities.

As such, as used herein, shielding electrodes can also be referred to as sorting electrodes or trapping electrodes if such electrodes are configured to participate in the sorting or trapping of discrete entities. Hence, shielding electrode 115a can also be referred to as a sorting electrode if it is configured to form a bipolar electrode pair with sorting electrode 103 to facilitate the sorting of discrete entities. Similarly, shielding electrode 115d can also be referred to as a trapping electrode if it is configured to form a bipolar electrode pair with trapping electrode 109 to facilitate the trapping of discrete entities.

In some cases, a shielding electrode can generate an electromagnetic field such that discrete entities in the device is at least partially shielded from undesired electromagnetic fields. Such undesired electromagnetic fields can originate from outside the microfluidic device or from within the microfluidic device. In some cases, the undesired electromagnetic fields are those fields that are not generated by a sorting electrode or by a trapping electrode. By at least partially shielding discrete entities in the microfluidic device, the shielding electrodes can inhibit the unintended merging of discrete entities (i.e. merging of discrete entities outside the discrete entity merger region). In some cases, shielding electrodes 115a, 115b, and 115c can be used to at least partially shield discrete entities from electromagnetic fields that are not generated by the sorting electrode or the trapping electrode.

In some cases, shielding electrodes can assist with the sorting of discrete entities. As an example, shielding electrode 115a can interact with sorting electrode 103 in order to facilitate sorting, such as by forming a bipolar electrode pair with sorting electrode 103. In some cases, sorting electrode 103 can be the charged electrode (e.g. positively charged), and shielding electrode 115a can be a ground. Stated in another manner, shielding electrode 115a can be configured to influence the shape of the electromagnetic field generated by sorting electrode 103 in order to facilitate sorting.

In some cases, shielding electrodes can assist with the trapping of discrete entities. As an example, shielding electrode 115d can interact with trapping electrode 109 in order to facilitate trapping, such as by forming a bipolar electrode pair with trapping electrode 109. In some cases, sorting electrode 109 can be the charged electrode (e.g. positively charged), and shielding electrode 115d can be a ground. Stated in another manner, shielding electrode 115d can be configured to influence the shape of the electromagnetic field generated by trapping electrode 109 in order to facilitate sorting.

In some cases, one or more of the shielding electrodes are separate elements, such as when all the shielding electrodes are separate elements. In some cases, one or more of the shielding electrodes are directly electrically connected. In some cases, one or more of the shielding electrodes are different regions of a single electrode, such as part of a single piece of metal. In some cases, one or more of the shielding elements are attached to ground.

As shown in FIG. 4, in some cases, the device includes one or more shielding electrodes. In some cases, the device includes zero shielding electrodes, such as when the discrete entities are sorted using a single sorting electrode and the discrete entities are trapped using a single trapping electrode.

As such, discrete entities are sorted and selectively combined within a microfluidic device (i.e., without leaving the microfluidic device). Stated in another manner, the discrete entities are sorted and combined without leaving microfluidic sized channels and regions.

In addition, the present disclosure provides examples of specific elements and steps that can be used with the described devices, systems, and methods. As reviewed above, the trapping element and the sorting element can be electrodes that exert a dielectrophoretic force on the discrete entity. In some cases, the electrodes are microfluidic channels containing a conductive material (e.g. salt water, liquid metal, molten solder, or a conductive ink to be annealed later). In some cases, the electrodes are patterned on the substrate of the microfluidic device (e.g. a patterned indium tin oxide (ITO) glass slide). In some cases, the trapping element includes two electrodes. In some cases, the trapping element is a selectively actuatable bipolar droplet trapping electrode. In some cases, the sorting element includes two electrodes. In some cases, the sorting element includes a selectively actuatable bipolar droplet sorting electrode.

In some cases, the sorting channel includes a partial height flow divider. In some cases, the sorting channel has a concentric or essentially concentric flow path and a portion of the sorting electrode is positioned at the center of the arc of the concentric or essentially concentric flow path.

In some embodiments, the discrete entity includes particles (e.g., cells, reagents for making oligo template structures, nickases, quenched oligonucleotide probes, etc.). In some embodiments, the discrete entity includes a chemical reagent (e.g. a lysing agent or a PCR reagent). In some embodiments, the discrete entity includes both a cell and a chemical reagent.

In some cases, the sorting is passive sorting. In some cases, the sorting is active sorting (i.e., the sorting element sorts a discrete entity into one of at least two locations based on a detected property of the discrete entity or a component within the discrete entity, such as a signal from cleaved oligonucleotide probe). In some cases, the detected property is an optical property (e.g., from a reporter dye no longer quenched) and the device further includes an optical detector (e.g. an optical detector configured to detect an optical property of a discrete entity or a component within in the inlet channel). In some cases, the optical property is fluorescence and the device further includes a source of excitation light. In some cases, the sorting is based on the detected fluorescence of a reporter dye that forms a FRET pair in a dead or dying cell with the dye already present in the dead or dying cell.

In some cases, the discrete entity merger region can include structural elements that are configured to aid in the trapping and combination of discrete entities therein. In some cases, such structural elements are configured to aid in such trapping and combining by changing the speed or direction of the flow of fluid through an area of the discrete entity merger region.

The present disclosure also provides methods of using systems that include a microfluidic device, e.g. as described above, and one or more additional components, e.g. (a) a temperature control module operably connected to the microfluidic device; (b) a detector configured to detect a discrete entity in the input channel, wherein the microfluidic device is configured to sort a discrete entity in the sorting channel based on the detection by the detector; (c) an incubator operably connected to the microfluidic device or a discrete entity maker; (d) a sequencer operably connected to the microfluidic device; (e) a device configured to make a plurality of discrete entities, wherein the device is located within the microfluidic device or separately from the microfluidic device; and (f) one or more conveyors configured to convey a particle (e.g. a cell, or a discrete entity), wherein the discrete entity can contain a particle in some cases, between any combination of: the incubator, device configured to make a plurality of discrete entities, the microfluidic device, the sequencer.

In some cases, the methods include controlling the temperature of the microfluidic device using a temperature control module operably connected to the microfluidic device. In some cases, the methods include detecting a discrete entity in the input channel of the microfluidic device (e.g. detecting an optical property of the discrete entity or a component therein), and sorting the discrete entity based on the detecting. In some cases, the method includes incubating cells in an incubator that is operably connected to discrete entity maker or a microfluidic device. In some cases, the method includes making discrete entities with a discrete entity maker, wherein the discrete entity maker is located within the microfluidic device or separate from the microfluidic device. In some cases, the method includes moving a discrete entity between components of the system (e.g. with one or more conveyors).

The present disclosure also provides steps that can be performed after the release of a combined microfluidic droplet from a discrete entity merger region. In some cases, the method includes recovering a component (e.g. a cell, a chemical compound or a combination thereof), from the combined discrete entity. In cases where a combined discrete entity includes one or more cells, the one or more cells can be analyzed, for example, as shown in FIG. 1 to detect target protein(s) that are secreted.

The present methods allow for the selective combination of two or more discrete entities without the need to accurately time the release or to accurately time the sorting of the two or more discrete entities. As such, in some cases, a first discrete entity (e.g., containing a first cell) is trapped in the discrete entity merger region before a second discrete entity (e.g., containing a second cell) to be combined therewith has entered the outlet channel after being sorted. In some cases, the second discrete entity has not entered the sorter channel, has not entered the inlet channel, or has not even been made when the first discrete entity is trapped in the discrete entity merger region.

The present methods allow for the sorting of discrete entities based on whether they contain the selective combination of only those discrete entities that contain the desired components. In some cases, the method involves creating 5 or more combined discrete entities per minute, including 10 or more, 25 or more, 50 or more, 75 or more, 100 or more, 150 or more, 200 or more, or 300 or more. In some cases, the method involves making 300 or more combined discrete entities per hour, including 1,500 or more, 3,000 or more, 4,500 or more, 6,000 or more, 9,000 or more, 12,000 or more, or 21,000 or more. In some cases, the sorting step is performed such that discrete entities are sorted at a rate of 0.01 Hz or more (e.g. 0.1 Hz or more, 1 Hz or more, 10 Hz or more, 100 Hz or more, 1 kHz or more, 10 kHz or more, or 30 kHz or more). In some cases, an electromagnetic sorter is used instead of a mechanical sorter (e.g. a valve, to allow for faster sorting rates). In some cases, the trapping and combining steps are performed such that a combined discrete entity is formed or released at a rate of 1 Hz or more, e.g. 10 Hz or more, 100 Hz or more, or 1,000 Hz or more.

In some cases, a discrete entity is flowed such that it reaches the discrete entity merger region between 0.1 ms to 1,000 ms after being sorted, such as between 1 ms and 100 ms, between 2 ms and 50 ms, and between 5 ms and 25 ms. In some cases, the first outlet channel is between 0.2 mm long and 5 mm long. In some cases, the first outlet channel has a dimension (e.g., width or height or diameter) of between 5 µm and 500 µm, such as between 10 µm and 100 µm. In some cases, the carrier fluid containing the discrete entities is flowed into the inlet channel at a rate of between 1 µl per hour and 10,000 µl per hour, such as between 10 µl per hour and 1,000 µl per hour, 25 µl per hour and 500 µl per hour, and between 50 µl per hour and 250 µl per hour. In some cases, the spacer fluid is injected at a rate of between 100 µl per hour and 20,000 µl per hour, such as 500 µl per hour and 5,000 µl per hour. In some cases, the bias fluid is injected at a rate of between 100 µl per hour and 20,000 µl per hour, such as 500 µl per hour and 5,000 µl per hour. In some cases, the fluid used to create cell-containing discrete entities has a concentration of between 1,000 cells per ml and 10,000,000 cells per ml, such as between 10,000 cells per ml and 1,000,000 cells per ml, and between 50,000 cells per ml and 200,000 cells per ml. In some cases, the discrete entities have a volume between 1 pl and 10,000 pl, such as between 10 pl and 1,000 pl, or between 50 pl and 500 pl.

In some cases, the one or more cells from a discrete entity or a combined discrete entity are cultured for at least 30 minutes or more, such as 1 hour or more, 6 hours or more, 12 hours or more, 24 hours or more, 3 days or more, or 7 days or more. In some cases, the device can continuously operate by selectively combining discrete entities for 10 minutes or more, such as 30 minutes or more, 45 minutes or more, 90 minutes or more, or 180 minutes or more. In some cases, the device can make at least 100 combined discrete entities while continuously operating, such as 1,000 combined discrete entities or more, 10,000 combined discrete entities or more, or 100,000 combined discrete entities or more.

In some cases, the methods include making one or more discrete entities, such as with a discrete entity maker. In such cases, the discrete entity maker can be part of the microfluidic device or separate from the microfluidic device as otherwise described herein. If the discrete entity maker is separate from the microfluidic device, the discrete entity maker can be operably connected to the microfluidic device (e.g., such that discrete entities can flow from the maker to the microfluidic device), or the discrete entities can be moved to the microfluidic device without the discrete entity maker and microfluidic device being operably connected. The systems and devices can include one or more discrete entity makers configured to form discrete entities from a fluid stream. Suitable discrete entity makers include selectively activatable droplet makers and the methods may include forming one or more discrete entities via selective activation of the droplet maker. The methods may also include forming discrete entities using a droplet maker, wherein the discrete entities include one or more entities which differ in composition. In some cases, the discrete entity maker comprises a T-junction and the method includes T-junction drop-making. In some cases, making the discrete entities includes a step of emulsification. In some cases, the discrete entity maker is made, in part or in whole, of a polymer. In some cases, one or more surfaces of the discrete entity maker are coated with a fluorosilane (e.g. such a discrete entity maker can be used when fluorinated fluids are passed through the discrete entity maker).

In some cases when multiple types of discrete entities are made (e.g., discrete entities that contain different contents, such as one dyed first cell and one with a second dyed cell), the contents can affect the ability of the discrete entity maker to successfully make the discrete entities. As such, in some cases, different conditions for the discrete entity maker are used to make a first group of discrete entities with first contents than for making a second group of discrete entities with second contents.

Aspects of the disclosed methods may include making discrete entities using one or more cells from a biological sample. In such cases, each discrete entity may contain zero, one, or more than one cell. In some cases, such discrete entities can be made by incorporating the biological sample, cells from the biological sample, lysate from cells of the biological sample, or any other sample derived from the biological sample into a mixed emulsion. In some cases, the method further includes separating one or more components of the biological sample or otherwise processing the biological sample (e.g. via centrifugation, filtration, and the like), before making the discrete entities.

In some cases, after the making of the discrete entities but before introducing the discrete entities to an inlet channel of a microfluidic device as described herein, the discrete entities can be further modified (e.g. by adding reagents for making oligo template structures, nickases, quenched oligonucleotide probes, a dyed cell, a reagent, a drug, a hydrogel, an extracellular matrix, a bead, a particle, a biological material, media, or a combination thereof). In some cases, the reagent reagents for making oligo template structures (e.g., first and second oligo-antibody conjugates), nickases, quenched oligonucleotide probes, a dyed cell, a primer, a probe, a lysing agent, a surfactant, a detergent, a barcode, an antibody, protein, enzyme, or a fluorescent tag. In some cases, the bead is an RNA capture bead. In some cases, the bead is an immunoassay bead. In some cases, the barcode is an oligonucleotide. In some cases, different types of discrete entities are labeled with different types of barcodes, fluorescent tags (e.g., on the oligonucleotide probe), or a combination thereof.

Fluorescent tags (on the oligo probe, and/or dying a cell generally) can be used to image a discrete entity or combined discrete entity in the discrete entity merger region. Fluorescent tags can also be used to identify the particular type of discrete entities that were combined to create a given combined discrete entity. As such, the properties of the combined discrete entity or component thereof can be correlated with the contents that were used to make the original discrete entities. As an example, different types of cells can be labeled with different fluorescent tags and incorporated into discrete entities. After such cell-containing discrete entities are combined with other discrete entities (e.g. containing other cells), the outcome of the combined discrete entities can be observed. As some of all of the original discrete entities can be labeled with fluorescent tags, the resulting combined discrete entity can have multiple fluorescent tags. In other cases, the combined discrete entity only has one fluorescent tag. Oligonucleotide barcodes can be used in a similar manner to that of fluorescent tags. Instead of detecting optical fluorescence, however, the oligonucleotide barcodes can be sequenced in order to identify the original discrete entities that formed the combined discrete entity.

Methods and devices which may be utilized in the encapsulating of a component from a biological sample are described in PCT Publication No. WO 2014/028378, the disclosure of which is incorporated by reference herein in its entirety and for all purposes. Encapsulation approaches of interest also include, but are not limited to, hydrodynamically-triggered drop formation and those described by Link, et al., Phys. Rev. Lett. 92, 054503 (2004), the disclosure of which is incorporated herein by reference. Other methods of encapsulating cells into droplets may also be applied. Where desired, the cells may be stained with one or more antibodies and/or probes prior to encapsulating them into drops.

One or more lysing agents may also be added to the discrete entities (e.g., droplets), containing a cell, under conditions in which the cell(s) may be caused to burst, thereby releasing their genomes and target proteins. The lysing agents may be added after the cells are encapsulated into discrete entities. Any convenient lysing agent may be employed, such as proteinase K or cytotoxins. In particular embodiments, cells may be co-encapsulated in drops with lysis buffer containing detergents such as Triton X100 and/or proteinase K. The specific conditions in which the cell(s) may be caused to burst will vary depending on the specific lysing agent used. For example, if proteinase K is incorporated as a lysing agent, the discrete entities (e.g., droplets), may be heated to about 37-60° C. for about 20 min to lyse the cells and to allow the proteinase K to digest cellular proteins, after which they may be heated to about 95° C. for about 5-10 min to deactivate the proteinase K. In certain aspects, cell lysis may also, or instead, rely on techniques that do not involve addition of lysing agent. For example, lysis may be achieved by mechanical techniques that may employ various geometric features to effect piercing, shearing, abrading, etc. of cells. Other types of mechanical breakage such as acoustic techniques may also be used. Further, thermal energy can also be used to lyse cells. Any convenient methods of effecting cell lysis may be employed in the methods described herein as appropriate.

One or more primers may be introduced into the discrete entities for each of the genes to be detected. Hence, in certain aspects, primers for all target genes (e.g., antibody genes) may be present in the discrete entity at the same time, thereby providing a multiplexed assay. The discrete entities may be temperature-cycled so that discrete entities will undergo PCR. In certain embodiments, rolling circle amplification (RCA)-based proximity ligation is employed.

In some embodiments, a surfactant may be used to stabilize the discrete entities. In some cases, the discrete entities or the associated emulsion lack a surfactant. Accordingly, a discrete entity may involve a surfactant stabilized emulsion. Any convenient surfactant that allows for the desired reactions to be performed in the discrete entities, may be used. In other aspects, a discrete entity is not stabilized by surfactants or particles. The surfactant used depends on a number of factors such as the oil and aqueous phases (or other suitable immiscible phases (e.g., any suitable hydrophobic and hydrophilic phases)) used for the emulsions. For example, when using aqueous droplets in a fluorocarbon oil, the surfactant may have a hydrophilic block (PEG-PPO) and a hydrophobic fluorinated block (Krytox® FSH). If, however, the oil was switched to be a hydrocarbon oil, for example, the surfactant would instead be chosen so that it had a hydrophobic hydrocarbon block, like the surfactant ABIL EM90. In selecting a surfactant, desirable properties that may be considered in choosing the surfactant may include one or more of the following: (1) the surfactant has low viscosity; (2) the surfactant is immiscible with the polymer used to construct the device, and thus it doesn’t swell the device; (3) biocompatibility; (4) the assay reagents are not soluble in the surfactant; (5) the surfactant exhibits favorable gas solubility, in that it allows gases to come in and out; (6) the surfactant has a boiling point higher than the temperature used for PCR (e.g., 95° C.); (7) the emulsion stability; (8) that the surfactant stabilizes drops of the desired size; (9) that the surfactant is soluble in the carrier phase and not in the droplet phase; (10) that the surfactant has limited fluorescence properties; and (11) that the surfactant remains soluble in the carrier phase over a range of temperatures. Other surfactants can also be envisioned, including ionic surfactants. Other additives can also be included in the oil to stabilize the discrete entities including polymers that increase discrete entity stability at temperatures above 35° C.

The discrete entities (e.g., microdroplets) described herein may be prepared as emulsions, such as an aqueous phase fluid dispersed in an immiscible phase carrier fluid (e.g., a fluorocarbon oil or a hydrocarbon oil) or vice versa. In some cases, the carrier fluid comprises a fluorinated compound. In some cases, the carrier fluid is an aqueous fluid. The nature of the microfluidic channel (or a coating thereon) (e.g., hydrophilic or hydrophobic), may be selected so as to be compatible with the type of emulsion being utilized at a particular point in a microfluidic workflow.

Emulsions may be generated using microfluidic devices. Microfluidic devices can form emulsions composed of droplets that are uniform in size. The microdroplet generation process may be accomplished by pumping two immiscible fluids, such as oil and water, into a junction. The junction shape, fluid properties (viscosity, interfacial tension, etc.), and flow rates influence the properties of the microdroplets generated but, for a relatively wide range of properties, microdroplets of controlled, uniform size can be generated using methods like T-junctions and flow focusing. To vary microdroplet size, the flow rates of the immiscible liquids may be varied since, for T-junction and flow focus methodologies over a certain range of properties, microdroplet size depends on total flow rate and the ratio of the two fluid flow rates. To generate an emulsion with microfluidic methods, the two fluids are normally loaded into two inlet reservoirs (syringes, pressure tubes) and then pressurized as needed to generate the desired flow rates (using syringe pumps, pressure regulators, gravity, etc.). This pumps the fluids through the device at the desired flow rates, thus generating microdroplet of the desired size and rate.

In some cases, a cell in a discrete entity may be labeled (e.g., by a fluorescent label, a barcode, or a combination thereof). In practicing the subject methods, a number of reagents may be incorporated into and/or encapsulated by, the discrete entities in one or more steps (e.g., about 2, about 3, about 4, or about 5 or more steps). Such reagents may include, for example, amplification reagents, such as Polymerase Chain Reaction (PCR) reagents. The methods of adding reagents to the discrete entities may vary in a number of ways. Approaches of interest include, but are not limited to, those described by Ahn, et al., Appl. Phys. Lett. 88, 264105 (2006); Priest, et al., Appl. Phys. Lett. 89, 134101 (2006); Abate, et al., PNAS, Nov. 9, 2010 vol. 107 no. 45 19163-19166; and Song, et al., Anal. Chem., 2006, 78 (14), pp 4839-4849; the disclosures of which are incorporated herein by reference. For instance, a reagent may be added to a discrete entity by a method involving merging a discrete entity with a second discrete entity which contains the reagent(s) in a discrete entity merger region of a microfluidic device described herein.

One or more reagents may also, or instead, be added using techniques such as droplet coalescence, or picoinjection. In droplet coalescence, a target drop may be flowed alongside a microdroplet containing the reagent(s) to be added to the droplet. The two droplets may be flowed such that they are in contact with each other, but not touching other microdroplets. These drops may then be passed through electrodes or other aspects for applying an electrical field, wherein the electric field may destabilize the microdroplets such that they are merged together. Reagents may also, or instead, be added using picoinjection. In this approach, a target drop may be flowed past a channel containing the reagent(s) to be added, wherein the reagent(s) are at an elevated pressure. Due to the presence of the surfactants, however, in the absence of an electric field, the microdroplet will flow past without being injected, because surfactants coating the microdroplet may prevent the fluid(s) from entering. However, if an electric field is applied to the microdroplet as it passes the injector, fluid containing the reagent(s) will be injected into the microdroplet. The amount of reagent added to the microdroplet may be controlled by several different parameters, such as by adjusting the injection pressure and the velocity of the flowing drops, by switching the electric field on and off, and the like.

In some cases, a discrete entity includes a bead. In some cases, at least one dimension of the bead (e.g., diameter, is between about 0.5 µm and about 500 µm). In some cases, the bead is made of a polymeric material, such as polystyrene. In some cases, the bead is magnetic or contains a magnetic component. In some cases, the bead has a biomolecule attached to its surface, such as an antibody, a protein, an antigen, DNA, RNA, streptavidin, or a combination thereof. In some cases, the bead is an immunoassay bead. In some cases, the bead is an RNA capture bead. As such, the present disclosure provides methods of selectively combining a biomolecule with another compound or cell, wherein the method includes selectively isolating the biomolecule from a composition using the bead, making a discrete entity that includes the bead and biomolecule, and selectively combining the discrete entity containing the bead and biomolecule with one or more other discrete entities that contain one or more other compounds or cells using the microfluidic device described herein. Methods of selectively isolating biomolecules using beads are known in the art, e.g. U.S. 2010/0009383, which is incorporated herein by reference for its disclosure of a method of separating a biomolecule or cell using beads.

In some embodiments, the methods, devices, and/or systems described herein can be used to sequence nucleic acid derived from single cells (e.g., once they have been determined to secrete the target protein, such as a monoclonal antibody). For example, individual cells can be encapsulated in the droplets which include the assay reagents as described herein. The cells can then be lysed and subjected to molecular biological processing to amplify and/or tag their nucleic acids with barcodes. The material from all the droplets can then be pooled for all cells and sequenced and the barcodes used to sort the sequences according to single droplets or cells. These methods can be used, for example, to sequence the genomes or transcriptomes of single cells in a massively parallel format.

In certain embodiments, nucleic acid sequence assay components that employ barcoding for labelling individual mRNA molecules, and/or for labeling for cell/well source (e.g., if wells pooled before sequencing analysis), and/or for labeling particular affixed entities (e.g., if droplet from two or more affixed entities are pooled prior to sequencing) are employed. Examples of such barcoding methodologies and reagents are found in Pat. Pub. US2007/0020640, Pat. Pub. 2012/0010091, U.S. Pat. 8,835,358, U.S. Pat. 8,481,292, Qiu et al. (Plant. Physiol., 133, 475-481, 2003), Parameswaran et al. (Nucleic Acids Res. 2007 Oct; 35(19): e130), Craig et al. reference (Nat. Methods, 2008, October, 5(10):887-893), Bontoux et al. (Lab Chip, 2008, 8:443-450), Esumi et al. (Neuro. Res., 2008, 60:439-451), Hug et al., J. Theor., Biol., 2003, 221:615-624), Sutcliffe et al. (PNAS, 97(5):1976-1981; 2000), Hollas and Schuler (Lecture Notes in Computer Science Volume 2812, 2003, pp 55-62), and WO201420127; all of which are herein incorporated by reference in their entireties, including for reaction conditions and reagents related to barcoding and sequencing of nucleic acids.

In certain embodiments, the DropSeq method employing beads with primers attached to them are employed to sequence nucleic acids from hybridomas. An example of such a method is described in Macosko et al., Cell, 161(5):1202-1214 (see, e.g., FIG. 1 therein), which is herein incorporated by reference in its entirety. In certain embodiments employing DropSeq, barcoded template switch oligos are bound to beads and oligo dT is supplied in solution along with RT PCR reagents. Reverse transcription (RT) can, for example, be performed as described in Kim et al., Anal Chem. 2018 Jan 16;90(2):1273-1279, herein incorporated by reference. In other embodiments, barcoded oligo-dT beads are provided, the cells are lysed, mRNAs is captured on the beads, the emulsion is broken, and the drop is re-emulsified to capture mRNA beads with barcoded TSO beads where the TSO can be released by UV. Solution phase TSO can then be used for performing RT-PCR. Primers specific to the variable regions displayed on the surface of the SD cells can be employed to amplify such variable regions prior to sequencing.

In certain embodiments, unique oligo drops are provided to the fixed entities, and allow a link between imaging and genomics. For example, the unique oligos can contain two part 8 mer barcodes linked to polyA or TSO followed by 8-mer barcodes. In this regard, if one employs 96 barcoded oligos, selecting any three can generate 142,880 combinations. It is known what combination of three oligos are printed at each well position to identify that particular well. These oligos will also be sequenced and so when one sees a particular 3-oligo combination in the sequencing readouts, one knows the fixed entity and the image for that fixed entity.

In certain embodiments, the barcode tagging and sequencing methods of WO2014201273 (“SCRB-seq” method, herein incorporated by reference) are employed. The necessary reagents for the SCRB-seq method (e.g., modified as necessary for small volumes) are added to the fixed entities, each containing a lysed cell. Briefly, the SCRB-seq method amplifies an initial mRNA sample from cells from a single fixed entity. Initial cDNA synthesis uses a first primer with: i) N6 for cell/well identification, ii) N10 for particular molecule identification, iii) a poly T stretch to bind mRNA, and iv) a region that creates a region where a second template-switching primer will hybridize. The second primer is a template switching primer with a poly G 3′ end, and 5′ end that has iso-bases. After cDNA amplification, the tagged cDNA single fixed entity samples are pooled. Then full-length cDNA synthesis occurs with two different primers, and full-length cDNA is purified. Next, a NEXTERA sequencing library is prepared using an i7 primer (adds one of 12 i7 tags to identify particular multi-well plates) and P5NEXTPT5 to add P5 tag for NEXTERA sequencing (P7 tag added to the other end for NEXTERA). The library is purified on a gel, and then NEXTERA sequencing occurs. As a non-liming example, with twelve i7 plate tags, and 384 cell/well-specific barcodes, this allows total of 4,608 single cell transciptomes to be done at once. This method allows for quantification of mRNA transcripts in single fixed entity.

In other embodiments, the barcode tagging and sequencing methods employ concepts from the Multi-seq method. For example, cells are incubated with anchor and co-anchor lipid modified oligonucleotides (LMO) and encapsulated in droplets. Individual barcodes in droplets can hybridize to exposed regions of the LMOs and these barcodes can be used instead of Dropseq beads. Anchor-coanchor LMOs remain bound to individual cells at 4° C. but can freely equilibrate between cells in a droplet at 37° C. Thus, a specific LMO-barcode combination in each droplet can be used to link two cells in that droplet that can be tracked after emulsion breaking. In one example, a unique LMO-barcode combination can be randomly assembled in every microfluidic droplet. Barcodes may also be deterministically pre-printed to a microwell array, and additionally provide linkage to imaging data recoded at specific microwell positions. In another embodiment, one cell in each combination may be LMO-barcoded before the combination in droplets. During incubation at 37° C., the LMO-barcodes will re-equilibrate to the initially non-barcoded cell and provide lasting information about co-encapsulation. If a unbarcoded B-cell is combined with an LMO-barcoded antigen presenting cell (APC), this process will allow the type of APC to be read out by sequencing only the B-cell.

In practicing the methods of the present disclosure, one or more sorting steps may be employed. A sorting step sorts a discrete entity into one of two or more locations (e.g. into one of two or more fluid channels). In some cases, the sorting is into one of two fluid channels. Discrete entities are sorted based on one or more properties of the discrete entity or a component within the discrete entity. In addition, such sorting may either be passive sorting or active sorting. Active sorting includes the detection of one or more properties of a discrete entity, or a component within the discrete entity, and sorting based on the detected property. Passive sorting involves sorting a discrete entity without the active detection of a property. Sorting approaches of interest include, by are not necessarily limited to, approaches that involve the use of one or more sorting channels and one or more sorting elements.

Sorting approaches which may be utilized in connection with the disclosed methods, systems and devices also include those described herein, and those described by Agresti, et al., PNAS vol. 107, no 9, 4004-4009.For active sorting, the device includes one or more sorting elements and one or more detectors, wherein each detector is configured to detect one or more properties of a discrete entity, or a component within the discrete entity, and each sorting element is configured to sort the discrete entity into one of two or more locations based on the detecting by the detection element. In some cases, a sorting element is positioned in proximity to the sorting channel, such as an electrode in proximity to the sorting channel. In some cases, a sorting element is positioned within the sorting channel, such as a partial height flow divider in a sorting channel. In some cases, the device includes a sorting element positioned within the sorting channel and one or more sorting elements positioned in proximity to the sorting channel. Exemplary structures and methods for active sorting discrete entities are described in Cole et al., PNAS, 2017, 114, 33, 8728-8733; Clark et al., Lab Chip, 2018, 5, 18, 710-713; and Sciambi et al., Lab on a Chip, 2015, 15, 47-51, the disclosures of which are incorporated herein by reference for sorting elements.

A variety of different components can be included in the discrete entities to facilitate detection, including one or more fluorescent dyes (e.g., as part of oligonucleotide probe and/or to stain cell(s)). Such fluorescent dyes may be divided into families, such as fluorescein and its derivatives; rhodamine and its derivatives; cyanine and its derivatives; coumarin and its derivatives; Cascade Blue and its derivatives; Lucifer Yellow and its derivatives; BODIPY and its derivatives; and the like. Exemplary fluorophores include indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX), LIZ, VIC, NED, PET, SYBR, PicoGreen, RiboGreen, and the like. Descriptions of fluorophores and their use, can be found in, among other places, R. Haugland, Handbook of Fluorescent Probes and Research Products, 9th ed. (2002), Molecular Probes, Eugene, Oreg.; M. Schena, Microarray Analysis (2003), John Wiley & Sons, Hoboken, N.J.; Synthetic Medicinal Chemistry 2003/2004 Catalog, Berry and Associates, Ann Arbor, Mich.; G. Hermanson, Bioconjugate Techniques, Academic Press (1996); and Glen Research 2002 Catalog, Sterling, VA.

In some embodiments, the microfluidic devices herein include directing the discrete entity to a discrete entity merger region. Accordingly, a device as described herein can include a discrete entity merger region and a trapping element positioned in proximity to the discrete entity merger region. The trapping element can trap a plurality of discrete entities in the discrete entity merger region for a time sufficient for the plurality of discrete entities to combine to form a combined discrete entity by exerting an electromagnetic force, exerting a mechanical force, applying heat, applying light, exerting an electrical force, providing a reagent, or a combination thereof sufficient. In some cases, the electromagnetic force is a dielectrophoretic force. In some cases, the electromagnetic force is an electrophoretic force. In some cases, the discrete entity merger region includes a feature selected from: a geometric change in a dimension of the first outlet channel, a flow obstacle, a flow divider, a laminating fluid inlet, a valve, or a combination thereof. In some cases, the geometric change is a change in the cross-sectional area of the first outlet channel (e.g., the discrete entity merger region has a larger cross-sectional area than the upstream region). In some cases, the geometric change is a change in one dimension of the first outlet channel (e.g., the discrete entity merger region is narrower than the downstream region). In some cases, the geometric change includes a recess in a channel wall. In some cases, the recess includes an area that is not colinear with the flow of fluid from the upstream region, such as shown as item 107 in FIG. 4. In some cases, where a valve is utilized, the valve is configured to switch between at least two states. In some cases, in the first state, the valve impedes the flow of a discrete entity past the discrete entity merger region while allowing flow of the carrier fluid past the discrete entity merger region. In some cases, in the second state, the valve is configured such that the combined discrete entity is not impeded from flowing past the discrete entity region. In some cases, the method includes putting the valve in a first state such that discrete entities can be trapped and combined into a combined discrete entity, and then putting the valve into a second state to release the discrete entity from the discrete entity merger region. In some cases, the valve is a membrane valve.

A laminating fluid inlet functions in a similar manner to certain embodiments of the spacer fluid inlet described above, such as a laminating fluid inlet is configured such that flowing fluid through the laminating fluid inlet will cause a discrete entity to move further away from a first side a channel and closer to a second side of a channel. Stated in another manner, the fluid flowing through the laminating fluid inlet contacts the fluid moving into the discrete entity merger region from an upstream region of the first outlet channel, thereby affecting the flow of fluid coming from the upstream region. In some cases, the fluid is oil, or a fluid which is otherwise immiscible with the fluid of the discrete entity.

FIG. 4 shows an embodiment wherein the discrete entity merger region includes recess 107, flow divider 113, and laminating fluid inlet 112. In FIG. 4, the laminating fluid provides a force pushing a discrete entity into recess 107 and towards trapping electrodes 109. In addition, flow divider 113 in FIG. 4 further affects the interaction of the laminating fluid and the fluid coming from the upstream region, thereby increasing the force pushing the discrete entity into recess 107. As such, a discrete entity merger region according to the present disclosure can include a laminating oil inlet and/or a flow divider, wherein such an element or elements are configured such that flowing oil through the laminating oil inlet channel will produce a force pushing a discrete entity in the discrete entity merger region towards a trapping electrode, a recess, or a combination thereof. In some embodiments, the device can include a flow divider without the laminating fluid inlet.

In some cases, the downstream region of the first outlet channel is configured to aid in the trapping of a discrete entity in the discrete entity merger region. In some cases, the downstream region has a larger cross-sectional area than the discrete entity merger region, which is an example of a geometric change in the first outlet channel. In some cases, the downstream region has a triangular or approximately triangular shape. In some cases, the downstream region has a triangular or approximately triangular shape and the discrete entity merger region is located at or near a vertex of the triangle. As an example, in the system of FIG. 5 has downstream region 208 and discrete entity merger region 207. In some cases, the longitudinal axis of the downstream region is parallel to the longitudinal axis of the discrete entity merger region, whereas in other cases such longitudinal axes are not parallel. In some cases, such axes are parallel but not colinear. In some cases, the axes are parallel and colinear. In some cases, the angle between such axes is greater than 0°, such as 5° or more, 10° or more, 15° or more, 30° or more, 45° or more, 60° or more, 75° or more, 90° or more, 135° or more, or 175° or more. In some cases, such an angle is between approximately 15° and approximately 135°. In some cases, such an angle is between approximately 60° and approximately 120°, such as shown in FIG. 5.

In some cases, the sorting element sorts discrete entities at a rate of at least 10 Hz, such as at least 100 Hz, at least 500 Hz, at least 1,000 Hz, at least 2,000 Hz, or at least 10,000 Hz. In some cases, only 50% or less of the discrete entities contain the contents desired for the second discrete entity, such as 25% or less, 10% or less, 5% or less, 1% or less, or 0.1% or less. In some cases, the discrete entity merger region and trapping element are configured to trap a first discrete entity for 0.1 ms or more, such as 1 ms or more, 5 ms or more, 10 ms or more, 25 ms or more, 50 ms or more, 100 ms or more, 500 ms or more, 1,000 ms or more, or 5,000 ms or more. In some cases, a first discrete entity is trapped in the discrete entity merger region for 0.1 ms or more before a second discrete entity enters the region, such as 1 ms or more, 10 ms or more, 100 ms or more, or 1,000 ms or more.

The present disclosure provides a method of performing reactions by selectively combining two or more discrete entities, as described above, wherein the reaction occurs between one or more components from each discrete entity (e.g., between a target and effector cell). Such components can be one or more cells, one or more products derived from a cell, one or more reagents (e.g., reagents for making oligo template structures, nickases, quenched oligonucleotide probes, etc.), or a combination thereof. In some cases, a suitable method includes combination of one cell and one or more reagents described herein. As an example, FIG. 6 shows the combination of four discrete entities, wherein three of the discrete entities each contain a different reagent or cell, and the fourth discrete entity contains a single cell. As such, FIG. 6 shows that a microfluidic device as described herein can be used to selectively combine different discrete entities, resulting in the formation of a combined discrete entity, e.g., that contains at least two cells and two reagents as described herein. In some cases, the reagents can include reagents for making oligo template structures, nickases, quenched oligonucleotide probes, cell lysing reagents, PCR reagents, reagents for analyzing the DNA or RNA within a cell, antibodies, or a combination thereof. In such cases, the method can further include collecting genomic data from contents of the discrete entities or combined discrete entities. In some cases, the one or more products derived from a cell include cell lysate, DNA, RNA, or a combination thereof. As such, the method can involve analyzing products from a cell, e.g. cell lysate, even though the cell per se is included in any of the discrete entities.

### EXAMPLES

### Example 1

### Example 1: Demonstration of Assay Compatibility With Different Nickase Enzymes

This Example demonstrates that multiple different nickases can generate signal under appropriate conditions. Instead of using antibody-oligo conjugates to form the template structure, a linear oligos were used as a proxies.

Method: Linear proxy templates and complementary probes were designed for the nickases Nt.BsmAI (FIG. 12, Panel A), Nb.BsrDI (FIG. 12, Panel B), Nb.BbvCI (FIG. 12, Panel C), and Nb.BtsI (FIG. 12, Panel D) as shown below:

Nt.BsmAI template: TTTACTGGAGAGACTTGAGACTC
GAACTTT (SEQ ID NO:1)

Nb.BsrDI template: TGGATGCTAGCAATGGTAACGATCGTAGGT
(SEQ ID NO:2)

Nb.BbvCI template: TTTTAGTGGTCCTCAGCTACTCGATGTTTT
(SEQ ID NO:3)

Nb.BtsI template: TTTTTGCCAGCAGTGAGTCAGACCGTTTTT (
SEQ ID NO:4)

Nt.BsmAI probe: FAM/AGTCTCAAGTCTCT/IABkFQ (SEQ ID NO:5)

Nb.BsrDI probe: FAM/ATCGTTACCATTGCTA/IABkFQ (SEQ ID NO:6)

Nb.BbvCI probe: FAM/GAGTAGCTGAGGAC/IABkFQ (SEQ ID NO:7)

Nb.BtsI probe: FAM/TCTGACTCACTGCT/IABkFQ (SEQ ID NO:8)

Proxy templates, probes, and nickase were mixed in buffer (Tris buffer, pH 8) and incubated at 37° C. Fluorescence was measured every two minutes over three hours. Results are shown in FIG. 12, which shows signal amplification using four different nickase enzymes with hairpin proxy templates.

### Example 2

### Comparison of Nickase Activity in Different Buffer vs. Media

In general, the homogenous assays in microdroplets herein will need to be compatible with cells in media. To this end, this Example tested two different nickases (Nt.BsmAI and Nb.BtsI) for their compatibly in media (specifically, in XVIVO) which can be used in droplets containing cells.

Method: For Nt.BsmAI, a hairpin proxy template (see Schematic in FIG. 13) was employed, along with a probe. These sequences for this template and probe are shown below:

Hairpin proxy template (SEQ ID NO:9):GCTGAGGTGGTGT
CacacgtctgcggTTTTTTccgcagacgtgtGACACCATTAGAGAC

Probe: FAM/CTAGCAGTCTCTAATACCTCAGCGCTAG/IABkFQ (SEQ ID NO:10)

For Nb.BtsI, the linear proxy template and probe are as shown above in Example 1. These enzymes were combined with their hairpin or linear proxy templates and probes in buffer or buffer containing 70% XVIVO. Fluorescence was measured every two minutes over three hours.

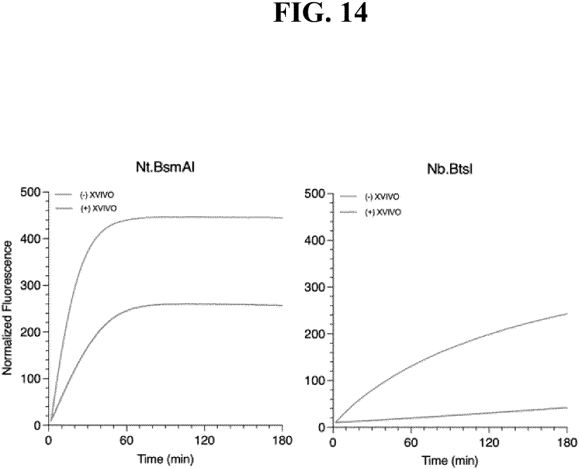

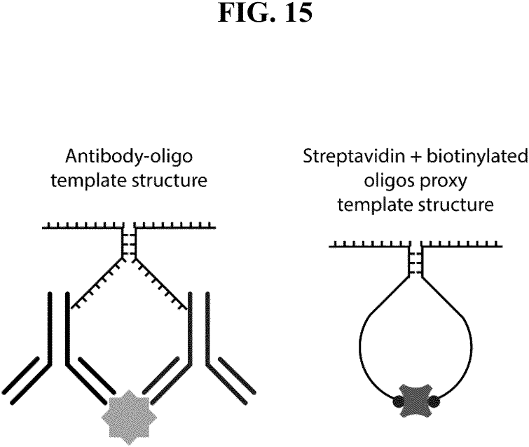

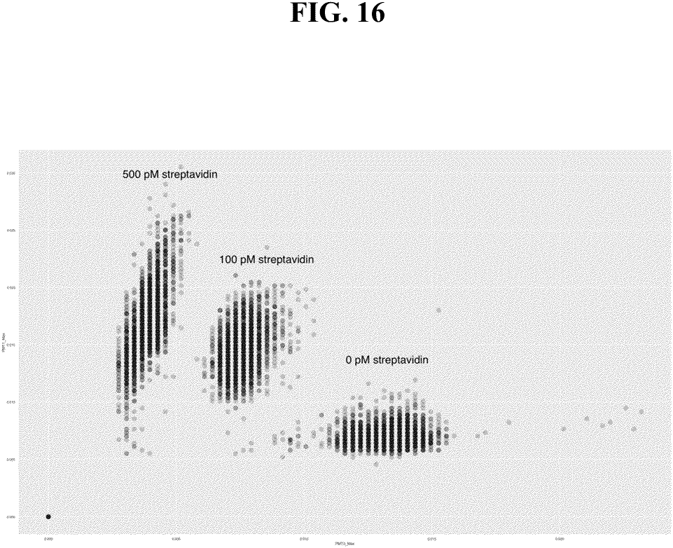

Results are shown in FIG. 14, which shows enzyme compatibility in media (XVIVO) for Nt.BsmAI (A) and Nb.Btsl (B).

### Example 3

### Demonstration of Proximity Based Signal Amplification in Droplets

This Example demonstrates that signal amplification and detection can occur in droplets using streptavidin as a target with two biotinylated oligos that when both bound to streptavidin can form the template structure.

Template arm 1: (SEQ ID NO: 11)
Biotin/TTTTTTTTTTTTTTTTTTTTTTTTTTTTTTTTTGACACCATTAGAGAC

Template arm 2: (SEQ ID NO: 12)
GCTGAGGTGGTGTCTTTTTTTTTTTTTTTTTTTTTTTTTTTTTTTTT/Biotin

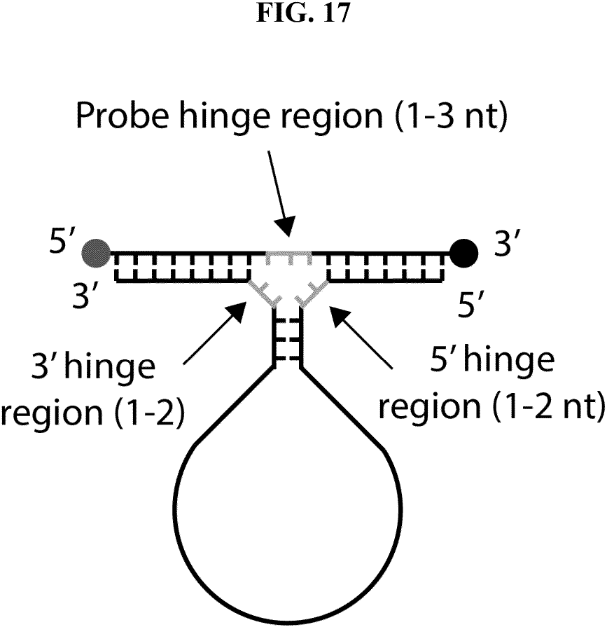

This is a proxy used in place of antibody-oligo reagents. This demonstration shows: 1) the nickase enzyme can work in droplets, 2) a proximity based method (two oligos binding streptavidin) can form the template structure and generate signal, and 3) fluorescence from droplets containing streptavidin can be detected and differentiated from background. FIG. 15 provides the antibody-oligo template structure vs. streptavidin/biotinylated oligos proxy.

Method: Droplets were prepared from solutions containing buffer, enzyme, biotinylated oligos, probe (detected on PMT1), and 0, 100, or 500 pM streptavidin. Each set of droplets was also encoded with a different concentration of drop detection dye (detected on PMT3). Fluorescence was then measured with a Scribe Microenvironment on Demand Platform in both the drop detection dye channel (PMT3) and the probe channel (PMT1). Droplets were prepared separately then mixed and analyzed together.

FIG. 16 shows successful detection of streptavidin in droplets using the biotinylated oligos.

### Example 4

### Demonstration of Tuning Signal Amplification Rate Based on Modification of Probe and Template Hinge Regions

This Example demonstrates that the signal amplification rate can be tuned by modifying the hinge regions of the probe and template arms.

Proxy O163: (SEQ ID NO: 13)GCTGAGGTGGTGTCTTTTTTTTT
TTTTTTTTTTTTTTTTTGACACCTTAGAGAC

Proxy O145: (SEQ ID NO: 14)GCTGAGGTGGTGTCTTTTTTTTT
TTTTTTTTTTTTTTTTTGACACCATTAGAGAC

Proxy O164: (SEQ ID NO: 15)GCTGAGGTGGTGTCTTTTTTTTT
TTTTTTTTTTTTTTTTTGACACCAATTAGAGAC

Proxy O165: (SEQ ID NO: 16)GCTGAGGTAGGTGTCTTTTTTTT
TTTTTTTTTTTTTTTTTTGACACCTTAGAGAC

Proxy O167: (SEQ ID NO: 17)GCTGAGGTAGGTGTCTTTTTTTT
TTTTTTTTTTTTTTTTTTGACACCATTAGAGAC

Proxy O173: (SEQ ID NO: 18)GCTGAGGTAGGTGTCTTTTTTTT
TTTTTTTTTTTTTTTTTTGACACCAATTAGAGAC

Proxy O166: (SEQ ID NO: 19)GCTGAGGTAAGGTGTCTTTTTTT
TTTTTTTTTTTTTTTTTTTGACACCTTAGAGAC

Proxy O174: (SEQ ID NO:20)GCTGAGGTAAGGTGTCTTTTTTTT
TTTTTTTTTTTTTTTTTTGACACCATTAGAGAC

Proxy O168: (SEQ ID NO:21)GCTGAGGTAAGGTGTCTTTTTTTT
TTTTTTTTTTTTTTTTTTGACACCAATTAGAGAC

Probe O161: (SEQ ID NO:22) FAM/CTAGCAGTCTCTAAACCTCAGCGCTAG/BHQ

Probe O183: (SEQ ID NO:23) FAM/CTAGCAGTCTCTAATACCTCAGCGCTAG/BHQ

Probe O185: (SEQ ID NO:24) FAM/CTAGCAGTCTCTAAAACCTCAGCGCTAG/BHQ

Probe O184: (SEQ ID NO:25) FAM/CTAGCAGTCTCTAATTACCTCAGCGCTAG/BHQ

Probe O162: (SEQ ID NO:26) FAM/CTAGCAGTCTCTAAAAACCTCAGCGCTAG/BHQ

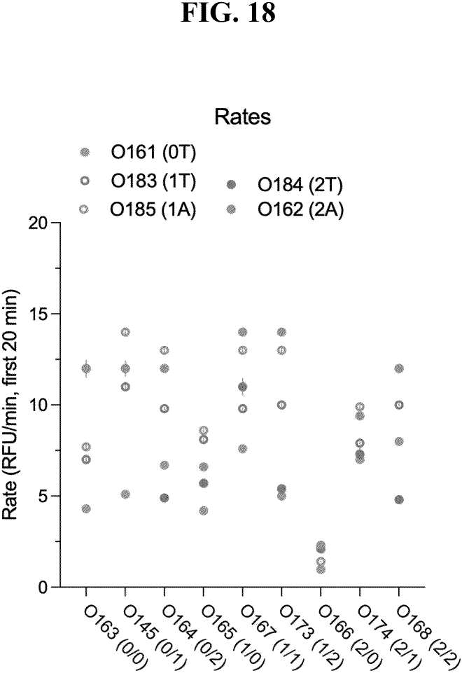

These are proxies used in place of oligo-antibody, and oligo-particle, reagents and different probes used to generate signal. This demonstration shows that by modifying the hinge region of the template arms and probe by inserting or removing nucleotides to make the arms and/or probe more or less flexible, the signal amplification rate can be tuned faster or slower. The hinge region refers to the sections of the template arms and probe that are not annealed when the probe is bound to the template (shown in orange in FIG. 17). Hinge regions can be made more or less flexible by adding nucleotides that are not matched to any corresponding nucleotide in the template arms/probe when the structure is annealed. FIG. 17 shows the location of the hinge regions in the template arms and probe.

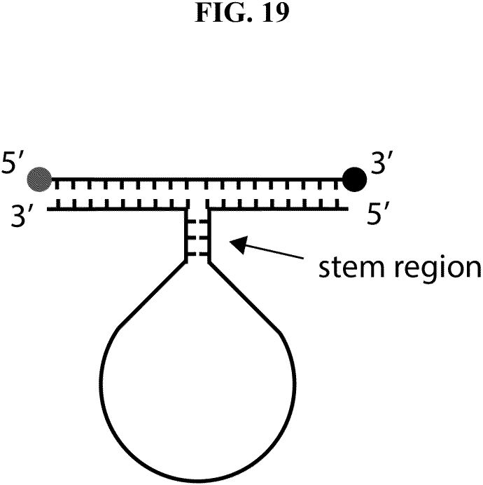

Method: A single proxy template was combined with a single probe and nickase in rCutSmart buffer (NEB) and incubated at 37° C. Fluorescence was measured every two minutes over three hours. Results are shown in FIG. 18, which shows average signal amplification rate over the first 20 min of the reaction. These results the beneficial effect of having nucleotides A or T in the 2 base probe region (e.g., AA, TT, AT, TA), and in the 5′ and 3′ hinge regions of the oligo-antibody or oligo-particle constructs.

### Example 5

### Demonstration of Signal Amplification From Templates With Varying Length Stem Regions

This example demonstrates that signal amplification is possible using templates with varying length stem regions or a template with no stem region.

Proxy O276: (SEQ ID NO:27) GCTGAGGTGGTGTCTTTTTTTTT
TGACACCTTAGAGAC

Proxy O277: (SEQ ID NO:28) GCTGAGGTGTGTCTTTTTTTTTT
GACACTTAGAGAC

Proxy O278: (SEQ ID NO:29) GCTGAGGTTGTCTTTTTTTTTTG
ACATTAGAGAC

Proxy O279: (SEQ ID NO:30) GCTGAGGTGTCTTTTTTTTTTGA
CTTAGAGAC

Proxy O280: (SEQ ID NO:31) GCTGAGGTTCTTTTTTTTTTGAT
TAGAGAC

Proxy O281: (SEQ ID NO:32) GCTGAGGTCTTTTTTTTTTGTTA
GAGAC

Proxy O282: (SEQ ID NO:33) GCTGAGGTTTTTTTTTTTTTAGA
GAC

Probe O184: (SEQ ID NO:34) FAM/CTAGCAGTCTCTAATTACCTCAGCGCTAG/BHQ

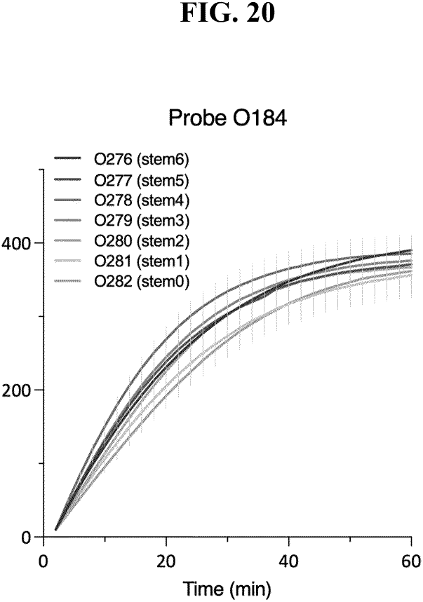

These are proxy templates used in place of antibody-oligo, or oligo-particle, reagents and a single probe used to generate signal. This demonstration shows that signal can still be generated using templates with shortened stem regions (e.g., 1, 2, 3, 4, 5, or 6 nucleotides) or no stem region at all. FIG. 19 shows the location of the stem region in the proxy template.

Method: A single proxy template was combined with probe, nickase, and Pluronic F-68 in rCutSmart buffer (NEB) and incubated at 37° C. Fluorescence was measured every two minutes over three hours. Results are shown in FIG. 20, which shows real time signal amplification curves for each sample.

### Example 6

### Demonstration of Signal Amplification From Different Template Structures

This example demonstrates that signal amplification is possible using templates with different structures, i.e. template structures that bind the probe in different ways.

Proxy O282: (SEQ ID NO:35) GCTGAGGTTTTTTTTTTTTTAGA
GAC

Proxy O285: (SEQ ID NO:36)TTAGAGACTTTTTTTTTTTTTTTT
TTTTTTTTTTTTTTGCTGAGGT

Proxy O288: (SEQ ID NO:37) ATCGGATCGCGTTTTTTTTTTTT
TTTTTTTTTTAGAGAC

Proxy O289: (SEQ ID NO:38) CGCGATCCGATTTTTTTTTTTTT
TTTTTTTTGCTGAGGT

Proxy O290: (SEQ ID NO:39) TTAGAGACTTTTTTTTTTTTTTT
TTTTTATCGGATCGCG

Proxy O291: (SEQ ID NO:40) GCTGAGGTTTTTTTTTTTTTTTT
TTTTTCGCGATCCGAT

Probe O287: (SEQ ID NO:41) FAM/AGTCTCTAATTACCTCAGC/BHQ

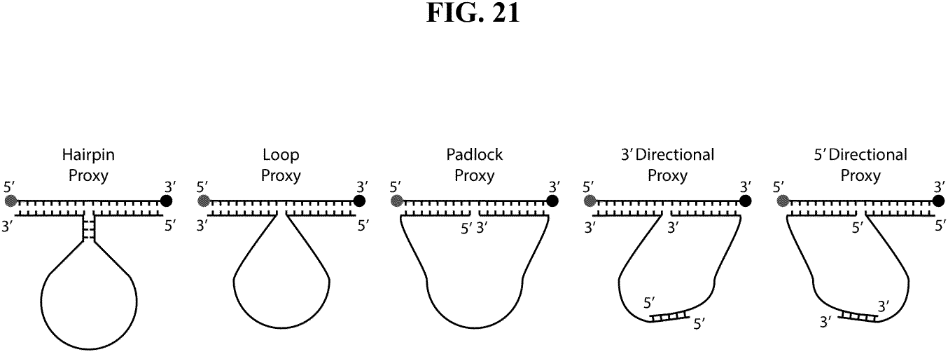

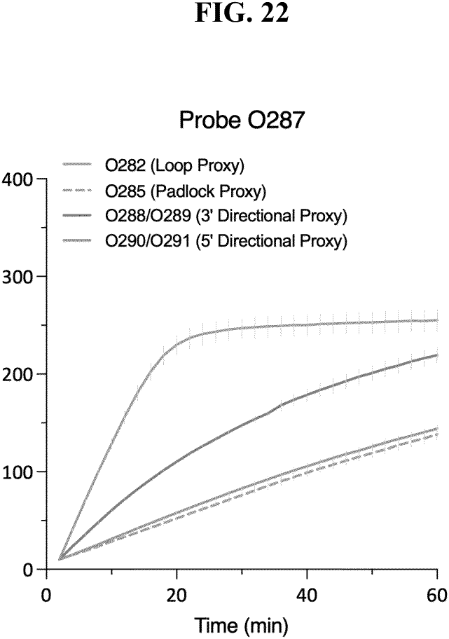

These are proxy templates used in place of antibody-oligo reagents and a single probe used to generate signal. This demonstration shows that signal can be generated using templates with alternative structures. FIG. 21 shows the structure of these templates and how they bind the probe.

Method: Proxy O282 or proxy O285 or proxy O288+proxyO289 or proxy 0290+proxy 0291 was combined with probe, nickase, and Pluronic F-68 in rCutSmart buffer (NEB) and incubated at 37° C. Fluorescence was measured every two minutes over three hours. Results are shown in FIG. 22, which shows real time signal amplification curves for each sample.

### Example 7

### Increased Nickase Concentration to Partially Overcome Effect of High Salt Concentration

In general, the homogenous assays in microdroplets herein should be compatible with cells in media. To this end, this Example tested different concentrations of nickase (Nt.BsmAI) in low salt assay buffer (20 mM Tris pH 7.4) and high salt assay buffer (20 mM Tris pH 7.4 + 137 mM NaCl). Tris + NaCl is a suitable media for short term cell culture.

Method: This example uses antibody-oligo reagents R77 and R78 designed to detect IFN-γ and an IFN-γ standard as the target. The reagents are described below.

R77 was synthesized by conjugating 0213 to a commercial anti-human IFN-γ antibody (Biolegend). R78 was synthesized by conjugating 0214 to a commercial anti-human IFN-γ antibody (Biolegend). Oligo sequences are listed below.

0213 (SEQ ID NO:42).TTTTTTTTTTTTTTTTTTTTTTTTTTTTTT
TTTTTGACACCATTAGAGAC

0214 (SEQ ID NO:43)GCTGAGGTAGGTGTCTTTTTTTTTTTTTTTT
TTTTTTTTTTTTTTTTTTT

Probe 0162 (SEQ ID NO:44)CTAGCAGTCTCTAAAAACCTCAGCG
CTAG

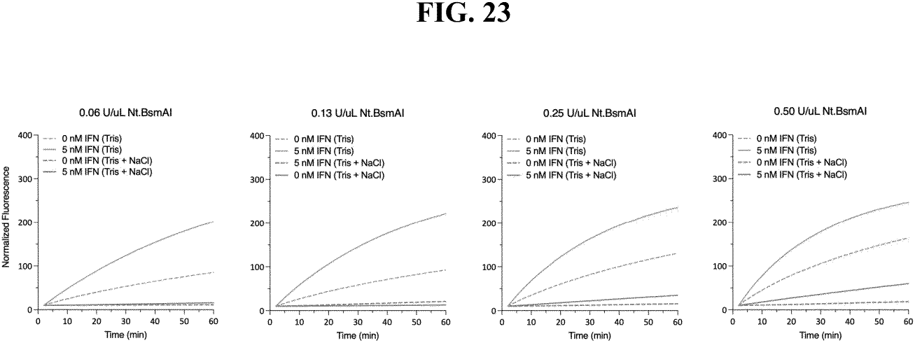

A mixture of R77 and R78 was combined with a buffer mix (20 mM Tris, 10 mM Mg, 0.1 mg/mL BSA, 0.05% Pluronic F-68), probe 0162 and either 0 nM or 5 nM IFN-γ in low or high salt buffer with varying concentrations of nickase present. Samples were incubated at 37 C and fluorescence was measured every two minutes over one hour. Results are shown in FIG. 23, which shows that higher enzyme concentration partially overcomes the inhibitory effect of high salt concentrations in the assay buffer.

### Example 8

### Detection of IFN-γ in Commercial Cell Media

In general, the homogenous assays in microdroplets herein will need to be compatible with cells in media. To this end, this Example tested detection of IFN-γ in 20% commercial cell media (Lonza XVIVO) + 60 mM NaCl. 20% XVIVO + 60 mM NaCl is a suitable media for short term cell culture. This example also demonstrates that both monoclonal and polyclonal antibodies can used for detection of IFN-γ in a homogeneous format.

Method: This example uses antibody-oligo reagents R79, R80, R113, and R114 designed to detect IFN-γ. IFN-γ standard is used as the target. The reagents are described below.

R79 was synthesized by conjugating 0211 to a commercial polyclonal anti-human IFN-γ antibody (Biolegend). R80 was synthesized by conjugating 0212 to a commercial polyclonal anti-human IFN-γ antibody (Biolegend). R113 was synthesized by conjugating 0211 to a commercial monoclonal anti-human IFN-γ antibody (Biolegend). R114 was synthesized by conjugating 0212 to a commercial monoclonal anti-human IFN-γ antibody (Biolegend). Oligo sequences are listed below.

0211 (SEQ ID NO:45)TTTTTTTTTTTTTTTTTTTTGACACCATTAG
AGAC

0212 (SEQ ID NO:46)GCTGAGGTAGGTGTCTTTTTTTTTTTTTTTT
TTTT

Probe 0162 (SEQ ID NO:44)CTAGCAGTCTCTAAAAACCTCAGCG
CTAG

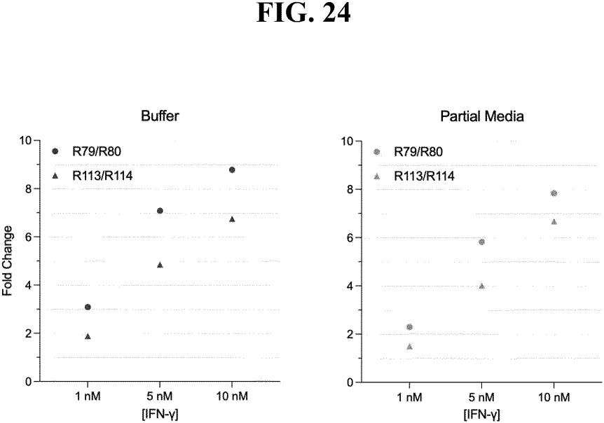

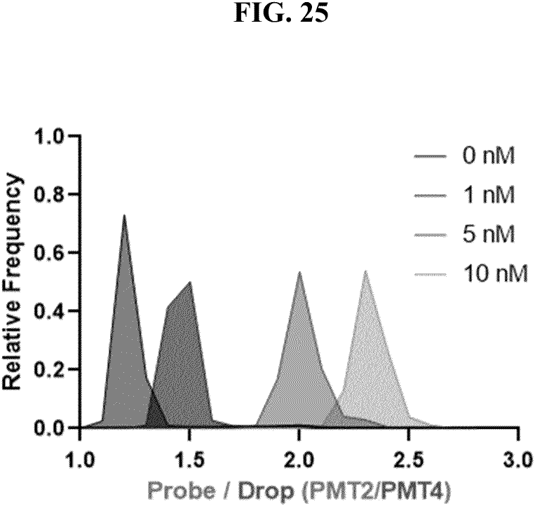

A mixture of R79 and R80 or a mixture of R113 and R114 was combined with a buffer mix (20 mM Tris, 10 mM Mg, 0.1 mg/mL BSA, 0.1% Tween 20, 20% XVIVO), probe 0162, nickase, and either 0, 1, 5, or 10 nM IFN-γ with 0 mM NaCl (“buffer”) or 60 mM NaCl (“partial media”) present. Samples were incubated at 37 C and fluorescence was measured every two minutes over one hour. Results are shown as fluorescence fold change at 60 min relative to 0 nM IFN-γ in FIG. 24. Results show that 5 and 10 nM IFN-γ can be detected using either polyclonal antibody-oligo reagents or monoclonal antibody-oligo reagents in cell-compatible partial media.

### Example 9

### Homogeneous Detection of IFN-γ in Microdroplets

This example demonstrates the detection of IFN-γ in assay buffer in the microdroplet environment.

Method: This example uses antibody-oligo reagents R77 and R78 designed to detect IFN-γ and an IFN-γ standard as the target. The reagents are described below.

R77 was synthesized by conjugating 0213 to a commercial anti-human IFN-γ antibody (Biolegend). R78 was synthesized by conjugating 0214 to a commercial anti-human IFN-γ antibody (Biolegend). Oligo sequences are listed below.

0213 (SEQ ID NO:42)TTTTTTTTTTTTTTTTTTTTTTTTTTTTTTT
TTTTGACACCATTAGAGAC

0214 (SEQ ID NO:43)GCTGAGGTAGGTGTCTTTTTTTTTTTTTTTT
TTTTTTTTTTTTTTTTTTT

Probe 0162 (SEQ ID NO:44)CTAGCAGTCTCTAAAAACCTCAGCG
CTAG

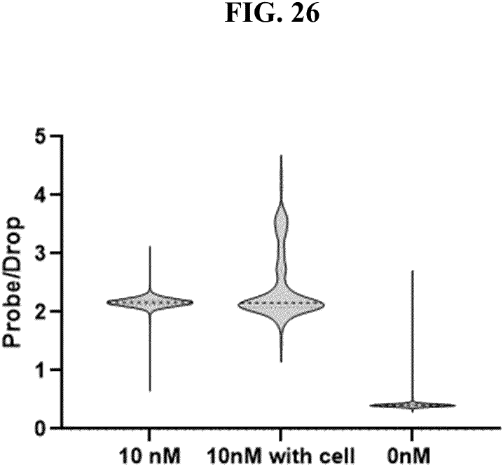

A mixture of R77 and R78 was combined with a rCutSmart buffer (NEB), Tween 20, probe 0162, nickase, and either 0, 1, 5, or 10 nM IFN-γ. A dextran-Alexa Fluor conjugate was also included in all samples for drop identification and normalization. Microdroplets were made from each sample using standard methods and were incubated at 37 C for two hours. After incubation, assay fluorescence in the droplets was measured on the Scribe instrument. Results are shown as assay fluorescence normalized to average droplet fluorescence in FIG. 25. Results show that 1, 5, and 10 nM IFN-γ can be detected in droplets using the homogeneous assay with antibody-oligo reagents. Concentrations listed in the legend are IFN-γ standard concentrations in the droplets.

### Example 10

### Homogeneous Detection of IFN-γ in Microdroplets Containing a Cell

This example demonstrates the detection of IFN-γ in microdroplets containing a cell.

Method: This example uses antibody-oligo reagents R113 and R114 designed to detect IFN-γ and an IFN-γ standard as the target. The reagents are described below.

R113 was synthesized by conjugating 0211 to a commercial monoclonal anti-human IFN-γ antibody (Biolegend). R114 was synthesized by conjugating 0212 to a commercial monoclonal anti-human IFN-γ antibody (Biolegend). Oligo sequences are listed below.

0211 (SEQ ID NO:45)TTTTTTTTTTTTTTTTTTTTGACACCATTAG
AGAC

0212 (SEQ ID NO:46)GCTGAGGTAGGTGTCTTTTTTTTTTTTTTTT
TTTT

Probe 0162 (SEQ ID NO:44)CTAGCAGTCTCTAAAAACCTCAGCG
CTAG

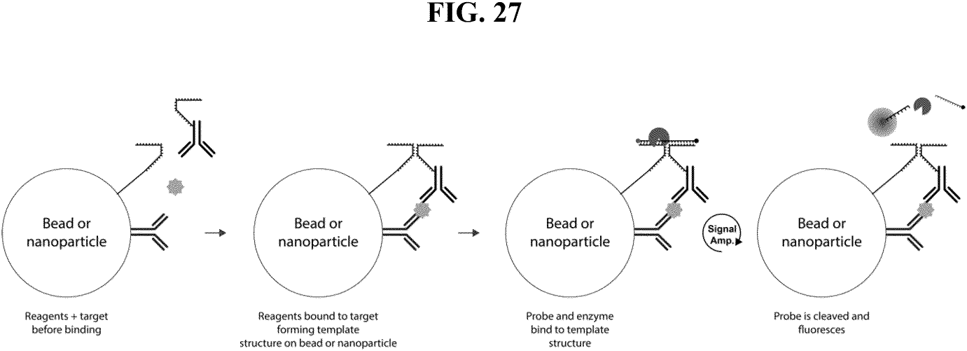

A mixture of R113 and R114 was combined with a buffer mix (20 mM Tris, 10 mM Mg, 0.1 mg/mL BSA, 0.1% Tween 20, 20% XVIVO, 60 mM NaCl), probe 0162, nickase, and either 0 or 10 nM IFN-γ. A dextran-Alexa Fluor conjugate was also included in all samples for drop identification and normalization. This mixture was combined with cells. Cells had been stained using a CellTracker dye for identification in microdroplets. Microdroplets were made from samples with and without cells using standard methods and were incubated at 37 C for three hours. After incubation, assay fluorescence, drop dye fluorescence, and cell fluorescence in the droplets was measured on the Scribe instrument. Results are shown as assay fluorescence normalized to average droplet fluorescence in FIG. 26. Assay fluorescence of droplets containing 0 and 10 nM IFN-γ standard plus a cell are compared to droplets containing 0 nM IFN-γ which do not contain a cell. Results show that the assay can detect 10 nM IFN-γ even when a cell is present in the droplet. Concentrations on the x-axis are IFN-γ standard concentrations in the droplets.

### Example 11

### Example of Assay Where Antibody-Antigen Sandwich Occurs on Bead or Nanoparticle

In this example the proximity assay occurs on the surface of a bead or nanoparticle. One of the capture antibodies or reagents is conjugated or bound to the bead or nanoparticle surface along with one of the oligos used to form the oligo template. The second antibody or reagent is conjugated or attached to the second oligo and is present in solution. When antigen is present it will bind both the antibody or capture reagent present on the bead and the antibody or capture reagent present in solution bringing the two oligos in proximity such that the oligo template is able to be formed. The beads used for this purpose can be a range of sizes, such as, for example, 10 nm - 10 um, and can be made from a range of materials. FIG. 27 shows a schematic of this form of the assay.

### Example 12

### Labelling Cell or Particle to Generate Homogeneous Signal in Droplet

In this example cells, beads, or particles (all generally referred to as particles) are labelled with an enzyme for which a suitable fluorogenic substrate exists. Labelling can occur via direct adsorption of the enzyme to the particle or through specific charge-based, oligo-based, antibody-based, or other particle-enzyme interaction. Enzyme can also be covalently linked to the particle through direct conjugation. Excess enzyme is then washed away prior to loading particles in droplets.

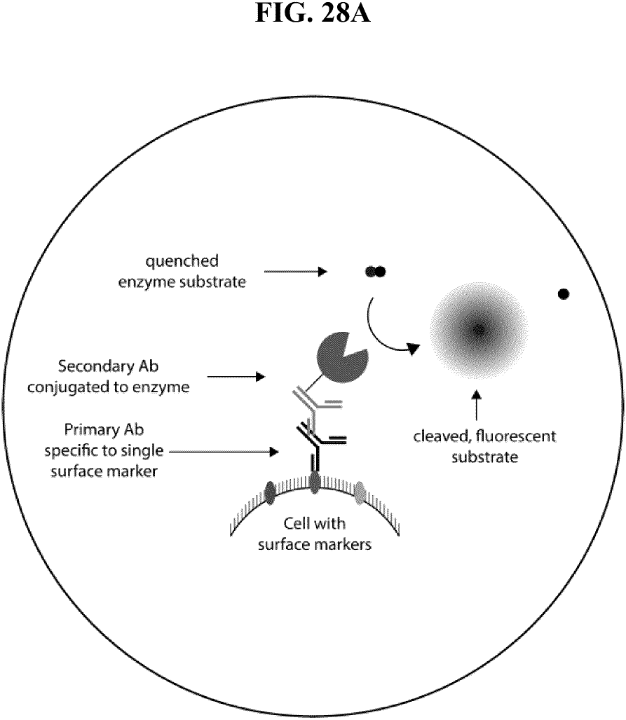

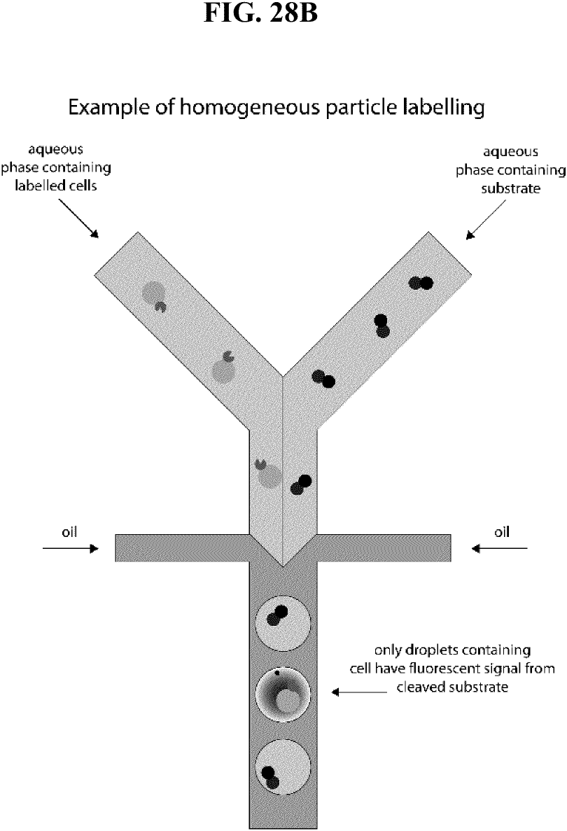

Droplets containing particles labelled with enzyme and containing fluorogenic substrate are created using a “co-flow” device in which two input solutions are used to create the dispersed phase. These two solutions are not mixed until just before droplet formation. The first input solution contains the labelled particles and the second input solution contains the fluorogenic substrate. Upon droplet formation, droplets containing a particle labelled with enzyme act on the fluorogenic substrate present in the droplet, generating a fluorescent signal. In this way, a homogeneous fluorescent signal is generated only in droplets containing a particle. FIG. 28A shows a schematic of droplet containing labelled cell using primary antibody against cell-surface marker and secondary antibody conjugated to enzyme. Fluorogenic substrate in the droplet is cleaved by the enzyme, generating a homogeneous fluorescent signal.

### Example 13

### Demonstration of Enzyme Buffer Compatibility for Homogeneous Labelling

This example demonstrates that two potential enzymes and their substrates which could be used for this labelling approach are compatible with multiple buffers commonly used when labelling cells or other particles.

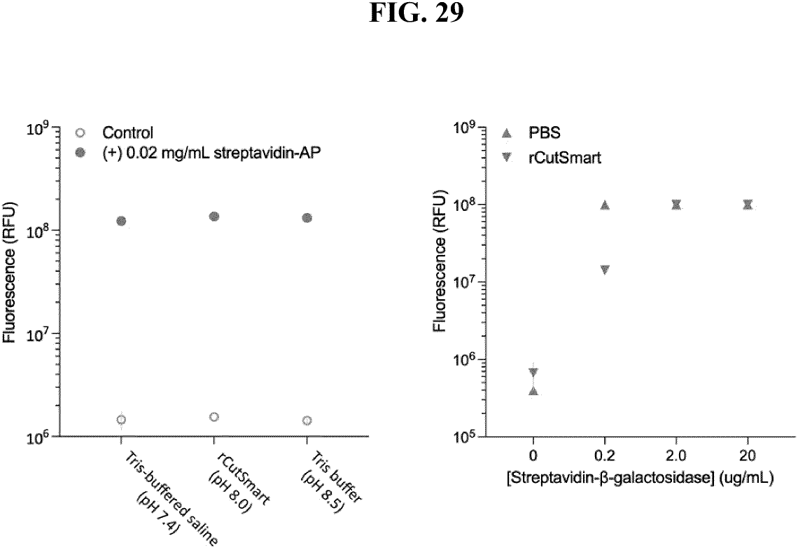

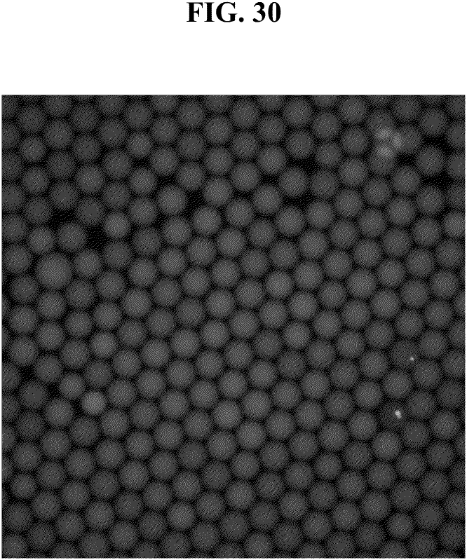

FIG. 29 shows plate reader data for two different potential enzymes that can be used. A streptavidin-alkaline phosphatase conjugate was mixed with 100 uM 6,8-difluoro-4-methylumbelliferyl phosphate (“DiFMUP”) in Tris-buffered saline (pH 7.4), rCutSmart buffer (pH 8.0), or Tris buffer (pH 8.5) and allowed to react for 5 min. Control solutions containing substrate but no streptavidin-alkaline phosphatase conjugate were also included. Fluorescence of each solution was then measured on a plate reader.

Similarly, different concentrations of a streptavidin-β-galactosidase conjugate were mixed with 100 uM 3-Carboxyumbelliferyl b-D-galactopyranoside (“CUG”) in phosphate-buffered saline (pH 7.4) or rCutSmart buffer (pH 8.0) and allowed to react for 5 min. Fluorescence of each solution was then measured on a plate reader.
Tris-buffered saline = 25 mM Tris-HCl, 2.7 mM KCl, 137 mM NaCl, pH 7.4
rCutSmart buffer = 20 mM Tris-acetate, 50 mM KOAc, 10 mM Mg(OAc)2, 100 ug/mL BSA, pH 7.9
Tris buffer = 10 mM Tris, 137 mM NaCl, pH 8.5
Phosphate-buffered saline = 25 mM Tris-HCl, 2.7 mM KCl, 137 mM NaCl, pH 7.4

### Example 14

### Labelling of Beads to Generate Homogeneous Signal in Droplet

This example demonstrates the labelling of beads with alkaline phosphatase such that droplets that contain a bead can be detected based on the homogeneous signal generated by the alkaline phosphatase-labelled bead.

Bead coated with biotin and Alexa Fluor 488 were labelled with a streptavidin-alkaline phosphatase then washed multiple times with PBS + 0.05% Tween 20 to remove excess streptavidin-alkaline phosphatase. Droplets were then created using a co-flow device with one aqueous solution containing 100 uM 6,8-difluoro-4-methylumbelliferyl phosphate (“DiFMUP”) and the second aqueous phase containing the alkaline phosphatase-labelled beads. 10 kDa Dextran-Alexa Fluor 647 was included with the labelled beads as a fixed fluorescent droplet dye. Beads were incubated for 15 min then imaged on a fluorescent microscope.

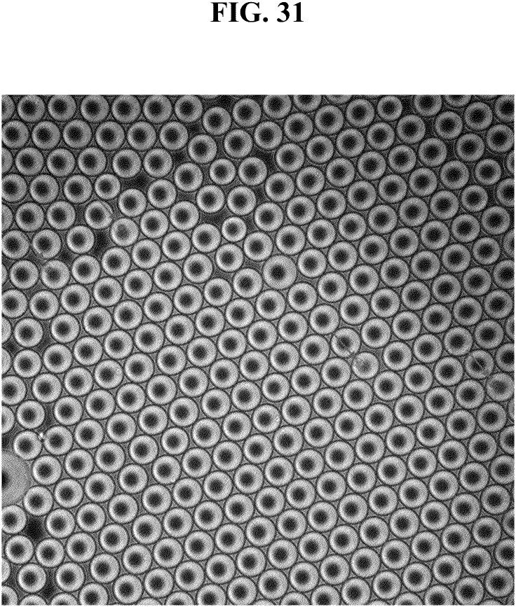

With this scheme, all droplets show Alexa Fluor 647 fluorescence (purple in FIG. 30), and droplets that contain labelled beads show 1) Alexa Fluor 488 fluorescence on the bead and 2) 405/448 ex/em fluorescence from the cleaved 6,8-difluoro-4-methylumbelliferyl phosphate (“DiFMUP”) substrate (blue in FIG. 30). FIG. 30 shows an image of beads generating a homogeneous signal in droplets.

### Example 15

### Labelling of Cells to Generate Homogeneous Signal in Droplet

This example demonstrates the labelling of cells with β-galactosidase such that droplets that contain a cell can be detected based on the homogeneous signal generated by the β-galactosidase-labelled cell.

Cells were first labelled with an anti-human CD5 mouse IgG2a antibody, washed with PBS/BSA, then labelled with an anti-mouse IgG secondary antibody conjugated to β-galactosidase. Droplets were then created using a co-flow device with one aqueous solution containing 100 uM 3-Carboxyumbelliferyl b-D-galactopyranoside (“CUG”) and the second aqueous phase containing the labelled cells. Cells were incubated for 15 min in droplets then imaged on a fluorescent microscope.

With this scheme droplets that contain labelled cells show 405/448 ex/em fluorescence from the cleaved 3-Carboxyumbelliferyl b-D-galactopyranoside (“CUG”) substrate (bright blue in figure). FIG. 31 shows labelled cells generating a homogeneous signal in droplets. Cells are denoted with red arrows.

### REFERENCES

1. Tan, Y., et al. (2014). “Proximity-dependent protein detection based on enzyme-assisted fluorescence signal amplification.” Biosens Bioelectron 51: 255-260.

2. Xue, L., et al. (2012). “Sensitive and homogeneous protein detection based on target-triggered aptamer hairpin switch and nicking enzyme assisted fluorescence signal amplification.” Anal Chem 84(8): 3507-3513.

3. https://www.abcam.com/content/fluorescence-resonance-energy-transfer-fret-assays

4. Hepojoki, S., et al. (2015). “Competitive Homogeneous Immunoassay for Rapid Serodiagnosis of Hantavirus Disease.” J Clin Microbiol 53(7): 2292-2297.

5. Romei, M. G. and S. G. Boxer (2019). “Split Green Fluorescent Proteins: Scope, Limitations, and Outlook.” Annu Rev Biophys 48: 19-44.

6. https://www.promega.com/products/immunoassay-elisa/lumit-immunoassays/?gclid=CjwKCAjwsNiIBhBdEiwAJK4khoLu9UzJm5CgNM7HkND6cuTDHj6P y-IFzAvqillnJKJOFznM6WhnFhoCphAQAvD_BwE

7. Li, J., et al. (2019). “Target-induced structure switching of aptamers facilitates strand displacement for DNAzyme recycling amplification detection of thrombin in human serum.” Analyst 144(7): 2430-2435.

8. Xie, Y., et al. (2020). “Proximity Binding-Triggered Assembly of Two MNAzymes for Catalyzed Release of G-Quadruplex DNAzymes and an Ultrasensitive Homogeneous Bioassay of Platelet-Derived Growth Factor.” Anal Chem 92(1): 593-598.

9. Zieba, A., et al. (2018). “In situ protein detection with enhanced specificity using DNA-conjugated antibodies and proximity ligation.” Mod Pathol 31(2): 253-263.

10. Deng, B., et al. (2014). “Assembly of multiple DNA components through target binding toward homogeneous, isothermally amplified, and specific detection of proteins.” Anal Chem 86(14): 7009-7016.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A composition, kit, or system comprising:
a) a discrete entity comprising:
i) an oligonucleotide probe comprising: A) a nucleic acid sequence comprising a first sequence and a second sequence; B) a dye, which is optionally a fluorescent dye; and C) a quencher molecule, wherein said quencher molecule is positioned such that is quenches signal from said dye; ii) at least one of the following:
A) a first antibody-oligonucleotide conjugate comprising: a first antibody or antigen binding fragment thereof, attached to, or operably linked to, a first oligonucleotide arm which comprises: I) a first region hybridizable to said first sequence, II) optionally a first template structure forming (TSF) region, and III) first linking region attached to, or operably linked to said first antibody or antigen binding fragment thereof;
B) a particle-oligonucleotide conjugate comprising: I) a particle, II) said first antibody or antigen binding fragment thereof, attached to, or operably linked to said particle, and III) said first oligonucleotide arm which is attached to, operably linked to, said particle;
iii) a second antibody-oligonucleotide conjugate comprising: a second antibody or antigen binding fragment thereof, attached to, or operably linked to, a second oligonucleotide arm which comprises: A) a second region hybridizable to said second sequence, B) optionally a second template structure forming (TSF) region, and C) second linking region attached to, or operably linked to, said second antibody or antigen binding fragment thereof,
wherein when said first antibody, or antigen fragment thereof, binds a first epitope of a target protein, and said second antibody, or antigen fragment thereof, binds said first epitope or a second epitope of said target protein in proximity to said first epitope this forms an oligonucleotide template structure (OTS), which is stabilized by said first TSF region hybridizing to said second TSF region if both are present;
wherein said OTS allows said oligonucleotide probe to hybridize to both said first and second regions; and
iii) a nickase enzyme, wherein said nickase enzyme cleaves said oligonucleotide probe when it is hybridized said OTS such that said fluorescent dye is released and is no longer quenched by said quencher molecule; and
b) a carrier fluid, wherein said discrete entity is present in said carrier fluid.

2. The composition, kit, or system of claim 1,
wherein said first oligonucleotide arm further comprises 1 or 2 hinge nucleotides contiguous with said first region that are not hybridized to any nucleotides in said OTS; and/or
wherein said a second oligonucleotide arm further comprises 1 or 2 hinge nucleotides contiguous with said second region that are not hybridized to any nucleotides in said OTS.

3. The composition, kit, or system of claim 1, wherein said carrier fluid comprises oil, and wherein said discrete entity further comprises an aqueous solution; and/or wherein said nickase enzyme is selected from the group consisting of: Nt.BsmAI, Nb.BsrDI, Nb.BbvCI, and Nb.BtsI.

4. The composition, kit, or system of claim 1, wherein said discrete entity further comprises cell media.

5. The composition, kit, or system of claim 1, wherein said discrete entity further comprises a first cell.

6. The composition, kit, or system of claim 5, wherein said first cell secretes, or can be induced to secrete, said target protein.

7. The composition, kit, or system of claim 5, wherein said discrete entity further comprises a second cell.

8. The composition, kit, or system of claim 1, wherein said discrete entity further comprises said target protein.

9. The system of claim 1, further comprising: a microfluidic device, and/or said particle comprises a bead or nanoparticle that optionally ranges in size from 10 nm - 10 um.

10. The system of claim 9, wherein said microfluidic device comprises:
i) an inlet channel,
ii) a sorting channel in fluid communication with said inlet channel,
iii) first and second outlet channels in fluid communication with said sorting channel, wherein said first outlet channel comprises a merger region,
iv) a sorting element positioned in proximity to the sorting channel, and
v) a trapping element positioned in proximity to said merger region.

11. The composition, system, or kit of claim 1, wherein said discrete entity is a droplet.

12. The composition, system, or kit of claim 1, wherein said discrete entity has a diameter of from about 1 µm to 1000 µm.

13. A method of detecting the presence or absence of a target protein in a discrete entity comprising:
a) generating a discrete entity in carrier fluid, wherein said discrete entity comprises:
i) a first cell that may secrete a target protein;
ii) an oligonucleotide probe comprising: A) a nucleic acid sequence comprising a first sequence and a second sequence; B) a fluorescent dye; and C) a quencher molecule, wherein said quencher molecule is positioned such that is quenches signal from said fluorescent dye;
iii) at least one of the following:
A) a first antibody-oligonucleotide conjugate comprising: a first antibody or antigen binding fragment thereof, attached to, or operably linked to, a first oligonucleotide arm which comprises: I) a first region hybridizable to said first sequence, II) optionally a first template structure forming (TSF) region, and III) first linking region attached to, or operably linked to, said first antibody or antigen binding fragment thereof;
B) a first particle-oligonucleotide conjugate comprising: I) a particle, II) said first antibody or antigen binding fragment thereof, attached to, or operably linked to said particle, and III) said first oligonucleotide arm which is attached to, operably linked to, said particle;
iv) a second antibody-oligonucleotide conjugate comprising: a second antibody or antigen binding fragment thereof, attached to, or operably linked to, a second oligonucleotide arm which comprises: A) a second region hybridizable to said second sequence, B) optionally a second template structure forming (TSF) region, and C) second linking region attached to, or operably linked to, said second antibody or antigen binding fragment thereof,
wherein said first antibody, or antigen fragment thereof, binds a first epitope of said target protein, and said second antibody, or antigen fragment thereof, binds said first epitope or a second epitope of said target protein in proximity to said first epitope thereby forming an oligonucleotide template structure (OTS), which is stabilized by said first TSF region hybridizing to said second TSF region if both are present;
wherein said OTS allows said oligonucleotide probe to hybridize to both said first and second regions; and
v) a nickase enzyme, wherein said nickase enzyme cleaves said oligonucleotide probe when it is hybridized to said OTS such that said fluorescent dye is released and is no longer quenched by said quencher molecule thereby generating said signal; and
b) detecting the presence or absence of said signal from said fluorescent dye.

14. The method of claim 13, wherein said first oligonucleotide arm further comprises 1 or 2 hinge nucleotides contiguous with said first region that are not hybridized to any nucleotides in said OTS; and/or wherein said a second oligonucleotide arm further comprises 1 or 2 hinge nucleotides contiguous with said second region that are not hybridized to any nucleotides in said OTS.

15. The method of claim 13, wherein A) said absence of said signal indicates said target protein is not present in solution in said discrete entity after being secreted by said first cell and/or is not expressed on the surface of said first cell, or B) said presence of said signal indicates said target protein is present in solution in said discrete entity after being secreted by said first cell and/or is expressed on the surface of said first cell.

16. The method of claim 13, further comprising: c) flowing said discrete entity in said carrier fluid in a microfluidic fluidic device, and d) sorting said discrete entity into a waste channel if said signal is not detected or into a keep channel if said signal is detected.

17. The method of claim 13, wherein said first cell secretes, or can be induced to secrete, said target protein.

18. The method of claim 13, wherein said discrete entity further comprises a second cell.

19. The method claim 13, wherein said discrete entity further comprises said target protein.

20. The method of claim 13, wherein said discrete entity flows in said carrier fluid in a microfluidic device.

21. The method of claim 20, wherein said microfluidic device comprises:
i) an inlet channel,
ii) a sorting channel in fluid communication with said inlet channel,
iii) first and second outlet channels in fluid communication with said sorting channel, wherein said first outlet channel comprises a merger region,
iv) a sorting element positioned in proximity to the sorting channel, and
v) a trapping element positioned in proximity to said merger region.

22. The method of claim 13, wherein said discrete entity is a droplet.

23. The method of claim 13, wherein said discrete entity has a diameter of from about 1 µm to 1000 µm.

24. A method of generating a plurality of discrete entities and detecting the presence or absence of a signal therefrom comprising:
a) flowing a first dispersed phase fluid in a first inlet channel of a co-flow micro-capillary droplet maker, wherein said first dispersed phase fluid contains particles labelled with an enzyme or a first oligonucleotide,
wherein said co-flow micro-capillary droplet maker comprises: i) said first inlet channel, ii) a second inlet channel, iii) a merger region, iv) at least one continuous phase inlet channel, v) a droplet generating region in fluid communication with said at least one continuous phase inlet channel and said merger region, vi) an outlet channel in fluid communication with said droplet generating region; and vii) a signal detecting element in proximity to said outlet channel;
b) flowing a continuous phase fluid in said at least one continuous phase inlet channel; and
c) flowing a second dispersed phase fluid in said second inlet channel of said co-flow micro-capillary droplet maker such that is merges with said first dispersed phase fluid at said merger region to create a mixed dispersed phase fluid,
wherein said mixed dispersed phase fluid flows into said droplet generating region such that a plurality of microdroplets are generated by said droplet generating region which flow in said continuous phase fluid into said outlet channel,
wherein said second dispersed fluid contains: i) a substrate that generates a signal when acted upon by said enzyme, or ii) a second oligonucleotide and an enzyme, wherein said second oligonucleotide hybridizes to said first oligonucleotide and comprises a dye and a quencher molecule that is positioned such that is quenches signal from said dye, and wherein said enzyme cleaves said second oligonucleotide when it is hybridized to said first oligonucleotide to generate a signal; and
d) detecting the presence or absence of said signal in each of said plurality of microdroplets using said signal detecting element.
